(19) United States
(12) Patent Application Publication
(10) Pub. No.: US 2025/0339257 A1
(43) Pub. Date: Nov. 6, 2025

(54) COMPLIANT GRAFT FOR REPLACING VESSEL SEGMENT

(71) Applicant: EDWARDS LIFESCIENCES CORPORATION, Irvine, CA (US)

(72) Inventors: Leonardo Paim Nicolau Da Costa, Tampa, FL (US); Emil Karapetian, Huntington Beach, CA (US)

(21) Appl. No.: 19/266,017

(22) Filed: Jul. 10, 2025

Related U.S. Application Data
(63) Continuation of application No. PCT/US2024/012070, filed on Jan. 18, 2024.
(60) Provisional application No. 63/481,114, filed on Jan. 23, 2023.
(60) Provisional application No. 63/481,946, filed on Jan. 27, 2023.

(51) Int. Cl. A61F 2/07; A61F 2/24

(52) U.S. Cl. CPC A61F 2/07; A61F 2/24; A61F 2210/0014; A61F 2230/0008; A61F 2230/001; A61F 2230/0091; A61F 2250/0039

(57) ABSTRACT

Devices, systems, and/or methods can treat aneurysms and/or other conditions, while providing compliance characteristics to fluid vessels. Examples of the present disclosure can include an implant device with one or more sealing/tube features configured to attach to a fluid vessel and a compliant structure coupled to the one or more sealing/tube features. The compliant structure can include a frame and/or covering configured to expand and contract radially based on pressure within the fluid vessel to provide a change in volume.

### RELATED APPLICATION(S)

This application is a continuation of International Patent Application No. PCT/US2024/012070, filed Jan. 18, 2024, which claims the benefit of U.S. Provisional Patent Application No. 63/481,114, filed on Jan. 23, 2023, and U.S. Provisional Patent Application No. 63/481,946, filed on Jan. 27, 2023, the complete disclosures of which are hereby incorporated by reference in their entireties.

### BACKGROUND

### Field

The present disclosure generally relates to the field of medical devices and methods for vascular repair.

### Description of Related Art

Aneurysms are permanent dilations in blood vessel walls due to weakened or abnormal tissue. In some instances, an aneurysm can rupture, causing internal bleeding that presents a serious risk to the patient, such as death. Aneurysms can occur in various parts of the body, including the aorta, brain, and elsewhere. Further, the aorta and other blood vessels are affected by other conditions that adversely affect the function of the blood vessels.

### SUMMARY

Described herein are devices, methods, and/or systems that treat aneurysms and other conditions in blood vessels while providing compliance characteristics. For example, devices of the present disclosure can include one or more sealing/tube features configured to attach to a fluid vessel and a compliant structure coupled to the one or more sealing/tube features. The compliant structure can include a frame and/or covering configured to expand and contract radially based on fluid pressure within the fluid vessel to provide a change in volume over the cardiac cycle. Such change in volume can allow the blood vessel to mimic compliance of a healthy blood vessel and/or otherwise promote blood flow during, for example, a phase of the cardiac cycle.

For purposes of summarizing the disclosure, certain aspects, advantages, and/or features are described. It is to be understood that not necessarily all such advantages may be achieved in accordance with any particular example. Thus, the disclosed examples can be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes. In addition, various features of different disclosed examples can be combined to form additional examples, which are part of this disclosure. Throughout the drawings, reference numbers may be reused to indicate correspondence between reference elements.

FIGS. 1A illustrates an example representation of a heart and associated vasculature having various features relevant to one or more examples of the present disclosure.

FIGS. 1B-1 illustrates an example healthy aorta.

FIGS. 1B-2 illustrates an example unhealthy aorta.

FIG. 2A illustrates a front view of an example implant device/system configured to be implanted a target site and provide compliant characteristics to a fluid vessel in accordance with one or more examples.

FIG. 2B illustrates a side view of the implant device of FIG. 2A in accordance with one or more examples.

FIGS. 2C-1, 2C-2, and 2C-3 illustrate various examples cross-sectional views through the compliant structure of the implant device of FIG. 2A in accordance with one or more examples.

FIG. 2D illustrates a perspective view of the implant device of FIG. 2A in accordance with one or more examples.

FIG. 3-1 illustrates a cross-sectional view of the compliant structure of the implant device of FIG. 2A in a non-expanded form having an oval shape in accordance with one or more examples.

FIG. 3-2 illustrates a cross-sectional view of the compliant structure of the implant device of FIG. 2A in an expanded form having a circle shape in accordance with one or more examples.

FIG. 4 illustrates various frame types and cross-sectional shapes that can be implemented for the compliant structure of the implant device of FIG. 2A in accordance with one or more examples.

FIG. 5 illustrates an implant device with a prosthetic valve in accordance with one or more examples.

FIG. 6 illustrates an example implant device that includes multiple tube portions coupled to multiple compliant structures in accordance with one or more examples.

FIG. 7 illustrates an example implant device including one or more branch tubes/tube portions/features that radially-project/extend off a main tubular portion of the implant device in accordance with one or more examples.

FIG. 8 illustrates the implant device of FIG. 7 with a compliant structure separated into multiple compliant structure segments to provide additional flexibility/mobility in accordance with one or more examples.

FIG. 9-1 illustrates an example implant device configured to seal within a fluid vessel to provide compliant characteristics to the fluid vessel in accordance with one or more examples.

FIG. 9-2 illustrates sealing features and/or tube portions of an example implant device implemented with one or more stent/frame structures in accordance with one or more examples.

FIG. 9-3 illustrates example frames/frame structures that can be implemented for a compliant structure, sealing feature(s), and/or tube portion(s) in accordance with one or more examples.

FIG. 10 illustrates the implant device of FIG. 9 implemented with two lower tubes/tube portions in accordance with one or more examples.

FIG. 11 illustrates a device implanted within example anatomy of the patient, namely a resected portion of the aorta, in accordance with one or more examples.

FIG. 12-1 illustrates a device implanted within example anatomy of the patient. namely an aneurysmal portion/section of the thoracic aorta, in accordance with one or more examples.

FIG. 12-2 illustrates a device implanted within example anatomy of the patient, namely an aneurysmal portion/section near the aortic bifurcation, in accordance with one or more examples.

FIG. 13 illustrates a flow diagram of a process for implanting a device using a more invasive/surgical procedure in accordance with one or more examples.

FIG. 14 illustrates a flow diagram of a process for implanting a device using an endovascular/minimally invasive procedure in accordance with one or more examples.

### DETAILED DESCRIPTION

The headings provided herein are for convenience and do not necessarily affect the scope or meaning of the subject matter.

Although certain examples are disclosed below, the subject matter extends beyond the specifically disclosed examples to other alternative examples and/or uses and to modifications and equivalents thereof. Thus, the scope of the claims that can arise here from is not limited by any of the examples described below. In any method or process disclosed herein, the acts or operations of the method or process can be performed in any suitable sequence and are not necessarily limited to any particular disclosed sequence. Various operations can be described as multiple discrete operations in turn, in a manner that can be helpful in understanding certain examples; however, the order of description should not be construed to imply that these operations are order dependent. Additionally, the structures, systems, and/or devices described herein can be embodied as integrated components or as separate components. For purposes of comparing various examples, certain aspects of these examples are described. Not necessarily all such aspects or advantages are achieved by any particular example. Thus, for example, various examples can be carried out in a manner that achieves or optimizes one advantage or group of advantages as taught herein without necessarily achieving other aspects or advantages as can also be taught or suggested herein.

Certain reference numbers are re-used across different figures of the figure set of the present disclosure as a matter of convenience for devices, components, systems, features, and/or modules having features that can be similar in one or more respects. However, with respect to any of the examples disclosed herein, re-use of common reference numbers in the drawings does not necessarily indicate that such features, devices, components, or modules are identical or similar. Rather, one having ordinary skill in the art can be informed by context with respect to the degree to which usage of common reference numbers can imply similarity between referenced subject matter. Use of a particular reference number in the context of the description of a particular figure can relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a,’ ‘10’ is the numeric portion and ‘a’ is the alphabetic portion), references in the written description to the numeric portion (e.g., ‘10’) can refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present disclosure to a feature ‘10’ can be refer to either an identified feature ‘10a’ in a particular figure of the present disclosure or to an identifier ‘10’ or ‘10b’ in the same figure or another figure, as an example.

Certain standard anatomical terms of location are used herein to refer to the anatomy of animals, and namely humans, with respect to various examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, these terms are used herein for case of description to describe the positional relationship between element(s)/structures(s), as illustrated in the drawings. Spatially relative terms are generally intended to encompass different orientations of the element(s)/structures(s), in use or operation, in addition to the orientations depicted in the drawings. For example, an element/structure described as “above” another element/structure can represent a position that is below or beside such other element/structure with respect to alternate orientations of the subject patient or element/structure, and vice-versa. Spatially relative terms, including those listed above, can be relative to a respective illustrated orientation of a referenced figure.

### Vascular Anatomy

Certain examples are disclosed herein in the context of vascular implant devices, and in particular, compliance implant devices implanted in the aorta. However, although certain principles disclosed herein can be particularly applicable to the anatomy of the aorta, the compliance implant devices in accordance with the present disclosure can be implanted in, or configured for implantation in, any suitable or desirable blood vessels or other anatomy, such as the inferior vena cava, etc.

The anatomy of the heart and vascular system is described below to assist in the understanding of certain concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves can be configured to open and close in response to a pressure gradient present during various stages of the cardiac cycle (e.g., relaxation and contraction) to at least partially control the flow of blood to a respective region of the heart and/or to blood vessels (e.g., ventricles, pulmonary artery, aorta, etc.). The contraction of the various heart muscles can be prompted by signals generated by the electrical system of the heart.

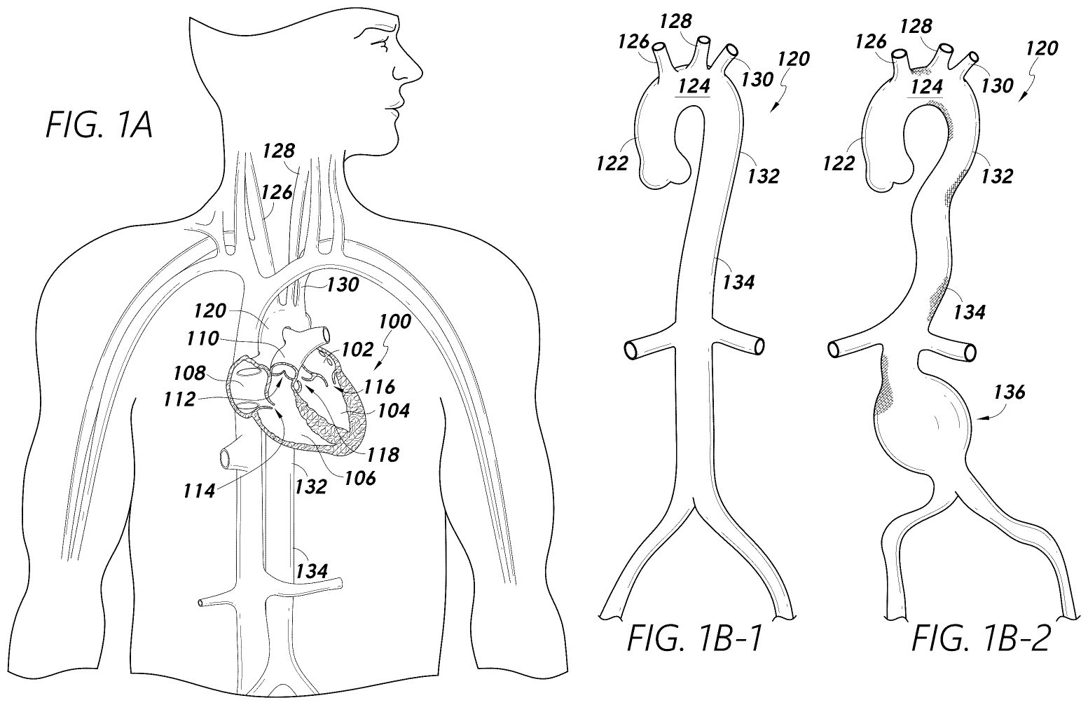

FIG. 1A illustrates an example representation of a heart 100 and associated vasculature having various features relevant to one or more examples of the present disclosure. The heart 100 includes four chambers, namely the left atrium 102, the left ventricle 104, the right ventricle 106, and the right atrium 108. In terms of blood flow, blood generally flows from the right ventricle 106 into the pulmonary artery 110 via the pulmonary valve 112, which separates the right ventricle 106 from the pulmonary artery 110 and is configured to open during systole so that blood can be pumped toward the lungs and close during diastole to prevent blood from leaking back into the heart from the pulmonary artery 110. The pulmonary artery 110 carries deoxygenated blood from the right side of the heart 100 to the lungs. The pulmonary artery 110 includes a pulmonary trunk and left and right pulmonary arteries that branch off the pulmonary trunk, as shown.

The tricuspid valve 114 separates the right atrium 108 from the right ventricle 106. The tricuspid valve 114 generally has three cusps/leaflets and can generally close during ventricular contraction (i.e., systole) and open during ventricular expansion (i.e., diastole). The mitral valve 116 generally has two cusps/leaflets and separates the left atrium 102 from the left ventricle 104. The mitral valve 116 is configured to open during diastole so that blood in the left atrium 102 can flow into the left ventricle 104, and, when functioning properly, closes during systole to prevent blood from leaking back into the left atrium 102. The aortic valve 118 separates the left ventricle 104 from the aorta 120. The aortic valve 118 is configured to open during systole to allow blood leaving the left ventricle 104 to enter the aorta 120, and close during diastole to prevent blood from leaking back into the left ventricle 104.

The heart valves can generally comprise a relatively dense fibrous ring, referred to herein as the annulus, as well as a plurality of leaflets or cusps attached to the annulus. Generally, the size of the leaflets or cusps can be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel can become dominant and press back against the leaflets. As a result, the leaflets/cusps come in apposition to each other, thereby closing the flow passage. Disfunction of a heart valve and/or associated leaflets (e.g., pulmonary valve disfunction) can result in valve leakage and/or other health complications.

The atrioventricular (mitral and tricuspid) heart valves generally are coupled to a collection of chordae tendineae and papillary muscles (not shown for visual clarity) for securing the leaflets of the respective valves to promote and/or facilitate proper coaptation of the valve leaflets and prevent prolapse thereof. The papillary muscles, for example, can generally comprise finger-like projections from the ventricle wall. The valve leaflets are connected to the papillary muscles by the chordae tendineae. A wall of muscle, referred to as the septum, separates the left 102 and right 108 atria and the left 104 and right 106 ventricles.

The vasculature of the human body, which can be referred to as the circulatory system, cardiovascular system, or vascular system, contains a complex network of blood vessels with various structures and functions and includes various veins (venous system) and arteries (arterial system). Generally, arteries, such as the aorta, carry blood away from the heart, whereas veins, such as the inferior and superior venae cavae, carry blood back to the heart.

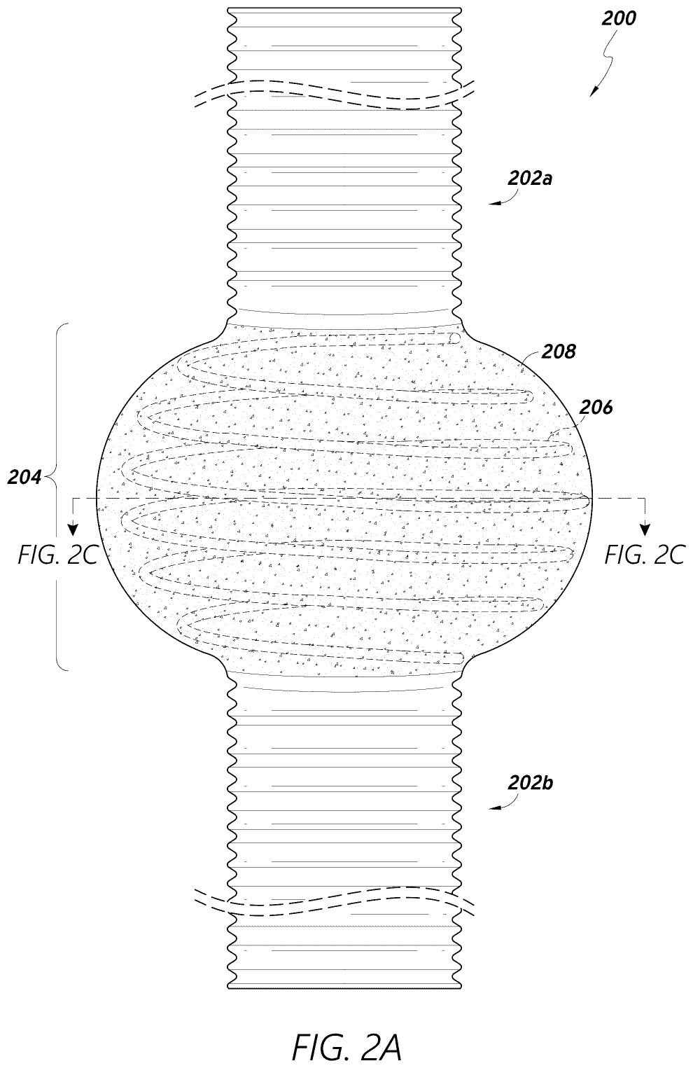

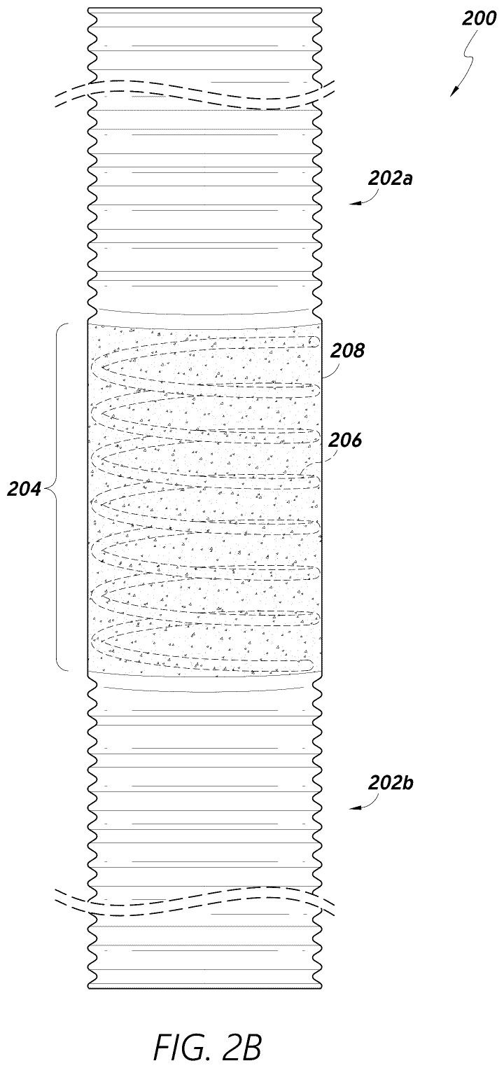

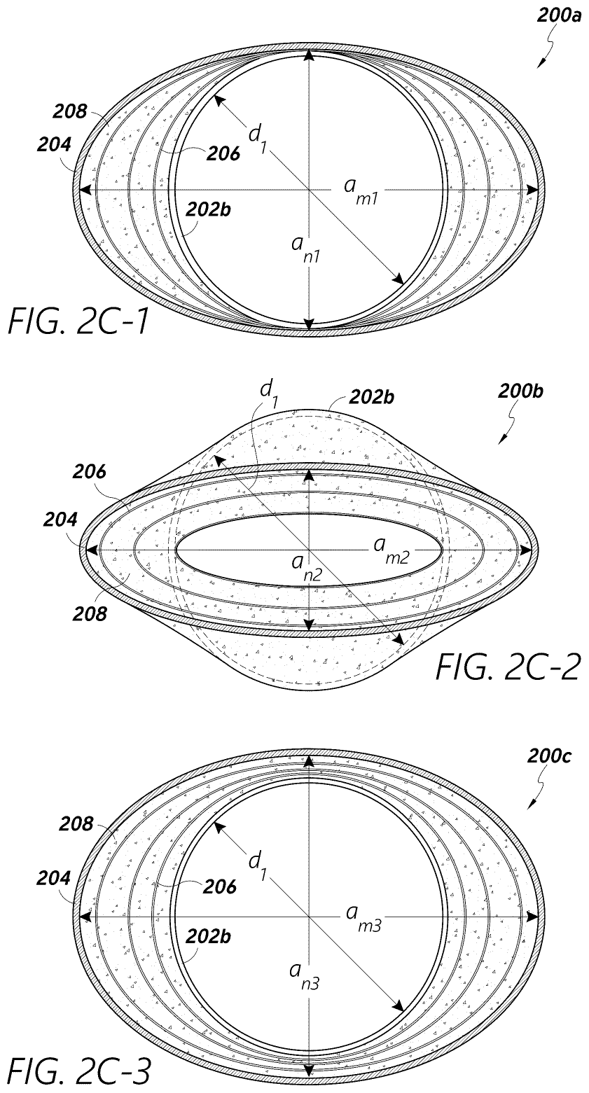

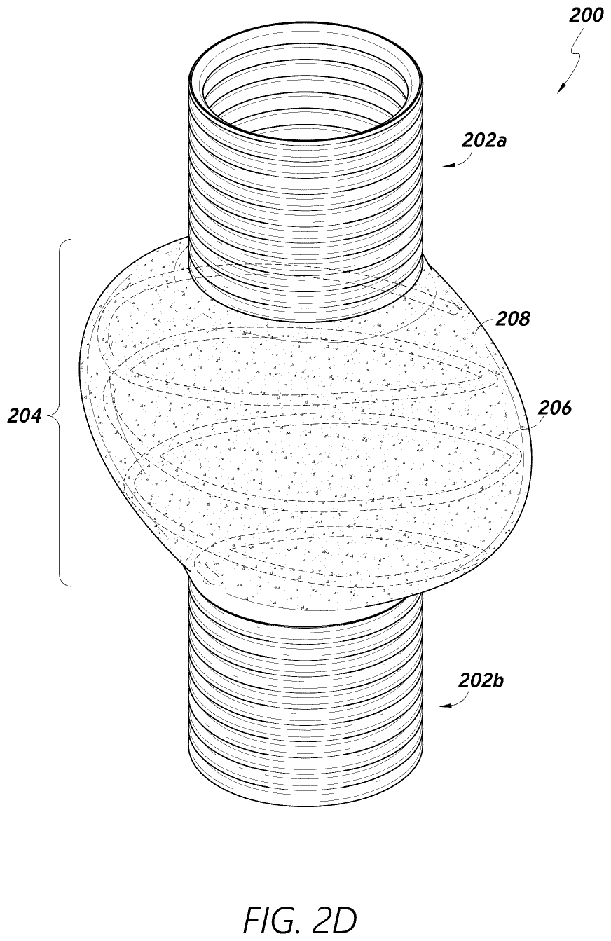

FIGS. 1B-1 and 1B-2 show detailed views of example healthy and unhealthy aortas 120, respectively. The aorta 120 is a compliant arterial blood vessel that buffers and conducts pulsatile left ventricular output and contributes the largest component of total compliance of the arterial tree. The aorta 120 includes the ascending aorta 122, which begins at the opening of the aortic valve 118 in the left ventricle 104 of the heart 100. The ascending aorta 122 and pulmonary trunk 110 twist around each other, causing the aorta 120 to start out posterior to the pulmonary trunk 110, but end by twisting to its right and anterior side. Among the various segments of the aorta 120, the ascending aorta 122 is relatively more frequently affected by aneurysms and dissections, often requiring open heart surgery to be repaired. The transition from ascending aorta 122 to aortic arch 124 is at the pericardial reflection on the aorta. At the root of the ascending aorta 122, the lumen has three small pockets between the cusps of the aortic valve 118 and the wall of the aorta 120, which are called the aortic sinuses or the sinuses of Valsalva. The left aortic sinus contains the origin of the left coronary artery and the right aortic sinus likewise gives rise to the right coronary artery. Together, these two arteries supply the heart with blood.

As mentioned above, the aorta 120 is coupled to the heart 100 via the aortic valve 118, which leads into the ascending aorta 122 and gives rise to the innominate artery 126, the left common carotid artery 128, and the left subclavian artery 130 along the aortic arch 124 before continuing as the descending thoracic aorta 132 and further the abdominal aorta 134. References herein to the aorta can be understood to refer to the ascending aorta 122 (also referred to as the “ascending thoracic aorta”), aortic arch 124, descending or thoracic aorta 132 (also referred to as the “descending thoracic aorta”), abdominal aorta 134, or other arterial blood vessel or portion thereof.

Arteries, such as the aorta 120, can utilize blood vessel compliance (e.g., arterial compliance) to store and release energy through the stretching of blood vessel walls. The term “compliance” can be used herein according to its broad and ordinary meaning, and can refer to the ability of an arterial blood vessel or prosthetic implant device to distend, expand, stretch, or otherwise deform in a manner as to increase in volume in response to increasing transmural pressure, and/or the tendency of a blood vessel (e.g., artery) or prosthetic implant device, or portion thereof, to recoil toward its original dimensions as transmural pressure decreases.

Arterial compliance facilitates perfusion of organs in the body with oxygenated blood from the heart. Generally, a healthy aorta and other major arteries in the body are at least partially elastic and compliant, such that they can act as a reservoir for blood, filling up with blood when the heart contracts during systole and continuing to generate pressure and push blood to the organs of the body during diastole. In older individuals and patients suffering from heart failure and/or atherosclerosis, compliance of the aorta and other arteries can be diminished to some degree or lost. Such reduction in compliance can reduce the supply of blood to the organs of the body due to the decrease in blood flow during diastole. Among the risks associated with insufficient arterial compliance, a significant risk presented in such patients is a reduction in blood supply to the heart muscle itself. For example, during systole, generally little or no blood can flow in the coronary arteries and into the heart muscle due to the contraction of the heart which holds the heart at relatively high pressures. During diastole, the heart muscle generally relaxes and allows flow into the coronary arteries. Therefore, perfusion of the heart muscle relies on diastolic flow, and therefore on aortic/arterial compliance.

A healthy aorta 120, as shown in FIG. 1B-1, runs along a generally straight path, whereas an aged and/or stiffened aorta 120, as shown in FIG. 1B-2, can run along a more tortuous, curved path. That is, the aorta tends to change in shape as a function of age, resulting in higher degrees of curvature or tortuosity, as developed gradually over time. Such change in shape of the blood vessel can be associated with the vasculature of the subject becoming less elastic. As such conditions develop, arterial blood pressure (e.g., left-ventricular afterload) can become more pulsatile, which can have deleterious effects, such as the thickening of the left ventricle (LV) muscle, and insufficient perfusion of the heart. Insufficient perfusion of the heart muscle can lead to and/or be associated with heart failure. Heart failure is a clinical syndrome characterized by certain symptoms, including breathlessness, ankle swelling, fatigue, and others. Heart failure may be accompanied by certain signs, including elevated jugular venous pressure, pulmonary crackles, and peripheral edema, for example, which may be caused by structural and/or functional cardiac abnormality. Such conditions can result in reduced cardiac output and/or elevated intra-cardiac pressures at rest or during stress.

As understood by those having ordinary skill in the art, the systolic phase of the cardiac cycle is associated with the pumping phase of the left ventricle, while the diastolic phase of the cardiac cycle is associated with the filling phase of the left ventricle. With proper arterial compliance, a change in volume will generally occur in an artery between high-and low-pressure phases of the cardiac cycle. With respect to the aorta, as blood is pumped into the aorta through the aortic valve, the pressure in the aorta increases and the diameter of at least a portion of the aorta expands. A first portion of the blood entering the aorta during systole may pass through the aorta during the systolic phase, while a second portion (e.g., approximately half of the total blood volume) may be stored in the expanded volume caused by compliant stretching of the blood vessel, thereby storing energy for contributing to perfusion during the diastolic phase. A compliant aorta may generally stretch with each heartbeat, such that the diameter of at least a portion of the aorta expands.

The tendency of the arteries to stretch in response to pressure as a result of arterial compliance can have a significant effect on perfusion and/or blood pressure in some patients. For example, arteries with relatively higher compliance can be conditioned to more easily deform than lower-compliance arteries under the same pressure conditions. Compliance (C) can be calculated using the following equation, where ΔV is the change in volume (e.g., in mL) of the blood vessel, and ΔP is the pulse pressure from systole to diastole (e.g., in mmHg):

$$C = \frac{Δ V}{Δ P} \qquad ( 1 )$$

A blood vessel that is relatively stiff can experience compliance that is diminished relative to a healthy blood vessel. Due to the stiffness of the blood vessel wall, the blood vessel can expand a relatively limited amount between diastole and systole. That is, during systole, the increased fluid pressure within the blood vessel can result in a relatively small and/or negligible expansion of the diameter of the blood vessel. Due to the limited expansion of the blood vessel, the change in volume in the blood vessel between phases of the cardiac cycle can likewise be limited, and therefore relatively little energy is stored in the blood vessel wall and returned to the blood circulation during low-pressure conditions, resulting in more pulsatile blood flow compared to healthy, compliant tissue.

Aortic stiffness and reduced compliance can lead to elevated systolic blood pressure, which can in turn lead to elevated intracardiac pressures, increased afterload, and/or other complications that can exacerbate heart failure. Aortic stiffness further can lead to reduced diastolic flow, which can lead to reduced coronary perfusion, decreased cardiac supply, and/or other complications that can likewise exacerbate heart failure.

FIG. 1B-2 illustrates the unhealthy aorta 120 with an aneurysm 136, which can include a permanent dilation/enlargement in the blood vessel due to weakened or abnormal tissue. Several sites for an aneurysm can include the Abdominal Aorta (e.g., Abdominal Aortic Aneurysm (AAA)), the ascending aorta, the aortic arch, the descending aorta, the thoracic aorta (e.g., Thoracic Aortic Aneurysm (TAA)), a portion of the aorta that spans several segments (e.g., Thoracoabdominal Aortic Aneurysm (TAAA)), and so on. In some instances, such as asymptomatic progressive aneurysmal dilation, an aneurysm can rupture, causing internal bleeding that presents a serious risk to the patient, such as death.

To treat an aneurysm in a blood vessel, a graft or other medical device can be implanted at the site of the aneurysm. For example, a physician can resect an aneurysmal portion of the aorta and implant a graft thereon. In various solutions, the graft includes a rigid structure, which can increase afterload for the left ventricle, cause long-term detrimental effects to the left ventricle, and/or cause other undesirable consequences.

Although the unhealthy aorta 120 is shown in FIG. 1B-2 with several undesirable characteristics including a more tortuous, curved path (in comparison to a healthy aorta) and an aneurysm 136, the unhealthy aorta 120 can additionally, or alternatively, include other issues/conditions. Further, although many examples are discussed in the context of aneurysms, the devices, methods, and/or systems disclosed herein can be implemented to treat a variety of conditions, such as stiffened blood vessels, acute aortic syndromes (AAS) (including aortic dissection (AD)), penetrating atherosclerotic ulcer (PAU), intramural hematoma (IMH), traumatic aortic injury (TAI), pseudoaneurysm, congenital abnormalities (including the coarctation of the aorta (CoA)), atherosclerotic and inflammatory affections, aortic rupture, genetic diseases (e.g. Marfan syndrome), and so on.

In view of the health complications that can be associated with aneurysms. reduced arterial compliance, and/or other conditions, it can be desirable in certain patients and/or under certain conditions, to treat the affected area and/or at least partially restore/alter compliance properties of the aorta or other blood vessels, or otherwise alter/control flow therein, in order to improve cardiac and/or other organ health.

### Implant Devices

The present disclosure relates to systems, devices, and methods for treating aneurysms and other conditions in blood vessels, such as the aorta or other arterial (or venous) vessel(s), while providing compliance characteristics. Examples of the present disclosure can include a device with one or more tube portions configured to attach to a fluid vessel and a compliant structure coupled to the one or more tube portions. The compliant structure can include a frame and/or covering configured to expand and contract radially based on pressure within the fluid vessel (e.g., luminal/radial pressure) to provide a change in volume over the cardiac cycle. Such change in volume can allow the device to mimic compliance of a healthy blood vessel and/or otherwise promote blood flow during, for example, a phase of the cardiac cycle. In instances, the cross-sectional shape of the device can change during expansion and contraction, such as from a first shape that has a smaller cross-sectional area to a second shape that has a larger cross-sectional area.

The systems, devices, and methods discussed herein can increase blood perfusion/flow and/or restore/provide compliance to the fluid vessels and/or other organs. For example, the compliant-enhancing devices can expand and store energy during higher-pressure periods of the cardiac cycle (e.g., during the systolic phase/period) and contract/compress during lower-pressure periods (e.g., during the diastolic phase/period) to return the stored energy to the circulation and increase flow through the vessel. As such, the devices can improve diastolic flow. Further, the systems, devices, and methods of this disclosure can avoid/minimize many of the negative effects that are commonly associated with implanting a medical device in a blood vessel. For example, the compliant-enhancing devices can mimic the expansion and contraction of a healthy blood vessel during phases of the cardiac cycle, which can avoid/minimize afterload to the heart (e.g., reduce left ventricle afterload), in comparison to other solutions that include relatively rigid structures. Moreover, the systems, devices, and methods can maintain a continuous flow pattern into the microvasculature of end-organs (e.g., minimize/avoid pulsatile flow), which can prevent end-organ damage. For example, cerebral, renal, coronary, etc. circulation can be improved.

The devices discussed herein can be implanted at an aneurysmal site or other target site using a variety of approaches, such as a surgical approach (e.g., open surgery) (which may resect a portion of the unhealthily blood vessel) and/or endovascular/minimally invasive approach. When implanted, the device can repair the aneurysmal site and/or otherwise provide an alternative/corrective blood channel through/around the aneurysm or other target site to improve perfusion of the blood through the vessel and/or other organ(s) of the body.

Methods and/or structures disclosed herein for treating a patient also encompass analogous methods and structures performed on or placed on a simulated patient, which is useful, for example, for training, demonstration, procedure and/or device development, and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof, synthetic, or any combination of natural and synthetic. Virtual elements can be entirely in silica or overlaid on one or more of the physical components. Virtual elements can be presented on any combination of screens, headsets, holographically, projected, loudspeakers, headphones, pressure transducers, temperature transducers, or using any combination of suitable technologies.

Implant devices, methods, and concepts disclosed herein can be described in the context of the aorta. However, such devices, methods, and/or concepts can be applicable in connection with any other artery or blood vessel.

FIGS. 2A, 2B, 2C-1 through 2C-3, and 2D illustrate front, side, cross-sectional, and perspective views, respectively, of an example device/system 200 (sometimes referred to as the “implant device 200”) configured to be implanted/disposed at a target site and provide compliant characteristics to a fluid vessel. The implant device 200 can generally include a tubular form that provides a fluid/blood channel/conduit to replace/substitute/support a native channel through the fluid vessel. The implant device 200 can include tubes/tube portions 202a, 202b (also referred to as “the tubular/luminal structures/features/elements 202”) coupled/attached or integral with a compliant structure 204 (also referred to as “the expandable/contractible/elastic structure/feature/component 204”). The tube portions 202 can be configured to couple/attach/contact a native fluid vessel (not illustrated), which can seal the implant device 200 to the native fluid vessel to provide a fluid path through the implant device 200 and connect to the native fluid vessel. The compliant structure 204 (and/or other portions of the implant device 200) can be configured to expand and/or contract radially based on luminal/radial pressure within the fluid vessel to provide a change in volume over the cardiac cycle.

The tube portions 202 can generally include a cross-sectional shape that matches or is otherwise similar to a cross-sectional shape of the fluid vessel in which the implant device 200 is implanted. For example, in the case of implanting the device 200 in the aorta, the tube portions 202 can include circle/circular cross-sectional shapes such that the tube portions 202 can fit to the cross-sectional shape of the aorta to form a tight seal therewith. However, the tube portions 202 can take any form/shape.

In examples, the tube portions 202 are less clastic than the compliant structure 204. Here, the tube portions 202 can be referred to as “non-compliant” or “more rigid” structures/sections/portion, whereas the compliant structure 204 can be referred to as a compliant section/portion. For instance, the tube portions 202 can be formed of a fabric/cloth that has elasticity characteristics below a threshold, while the compliant structure 204 can have clastic characteristics above the threshold. However, the tube portions 202 can have similar or greater elasticity characteristics than the compliant structure 204. In some cases, the tube portions 202 are formed of polyethylene terephthalate or another polyester. The tube portions 202 can be formed of a material that can be cut to adjust a size/length of the implant device 200 for the particular application. In examples, the tube portions 202 are formed of a material that is easily cut and/or minimizes fraying.

The compliant structure 204 can include a frame 206 (sometimes referred to as “the expandable frame 206”) and a covering/cover 208 disposed on the frame 206. The first tube portion 202a is coupled to a first end of the compliant structure 204 and the second tube portion 202b is coupled to a second end of the compliant structure 204. The covering 208 can be disposed around and/or within the inside of the frame 206, such that the covering 208 generally contacts and conforms to the shape/form of the frame 206 (e.g., expands and contracts along with the frame 206). The covering 208 can be elastic (or include certain elasticity characteristics/properties) to allow the covering 208 to expand and contract with the frame 206. The covering 208 can be coupled to the tube portions 202 to form a fluid tight seal with the tube portions 202 and provide a conduit for fluid flow through the implant device 200. The covering 208 can be formed of a material/cloth that is able to withstand many cycles without tearing/rupturing or otherwise being damaged. In examples, the covering 208 is formed of a plastic, such as a thermoplastic polyurethane (TPU) or another material. However, various materials can be used for the covering 208. In some cases, the tube portions 202 (or materials/coverings for the tubes 202) are integral with the covering 208 to form a continuous piece. In other cases, the tube portions 202 and covering 208 are separate components that are coupled together. Although various figures illustrate the covering 208 as being disposed around the frame 206, the covering 208 can be disposed within the frame 206 (i.e., the covering 208 is internal relative to the frame 206). The covering 208 can cover one or more internal and/or external portions/surfaces of the frame 206. For instance, the covering 208 can include a fabric/material with different layers, wherein the frame 206 can be embedded between different layers of the fabric/material. In some cases, the covering 208 can promote tissue ingrowth within the native blood vessel.

The compliant structure 204 is generally configured to expand and contract in at least one dimension based in part on fluid pressure associated with the fluid vessel in which the implant device 200 is implanted. The frame 206 (and/or covering 208 in some cases) can generally be biased to a particular shape/form (also referred to as “a biased/contracted state/form”), wherein such biased shape/form is associated with less volume/cross-sectional area than a non-biased form. As luminal pressure within the implant device 200 increases, the frame 206 and/or covering 208 can expand to the non-biased shape/form (also referred to as “an expanded state/form” or “secondary state/form”). The frame 206 can include shape memory/super elasticity to implement the biased form/shape. For instance, the frame 206 can be formed of nitinol or other shape-memory metal or material, which can allow the frame 206 to expand and return to the biased form. FIGS. 2A, 2B. 2C-1, 2C-2, 2C-3, and 2D illustrate the implant device 200 in a biased/non-expanded state. Although various figures show the implant device 200 with the frame 206, the implant device 200 can be implanted without a frame, such as by using just an elastic/flexible tube or another structure that facilitates expansion and contraction.

The terms “shape memory,” “shape memory effect,” “shape memory characteristic,” and the like are used herein according to their broad and ordinary meanings, and can refer to, for example, any tendency of a material, once deformed, remodeled, adjusted, or otherwise manipulated or configured from an original and/or set/biased shaped thereof, to return to the original/set/biased shape, form, or structure when a deforming force is removed or reduced. For example, in some contexts, shape memory or the like can relate or refer to the ability of a material/element to deform at a temperature when an external force is applied, maintain the deformed shaped when the external force is removed, and return to the undeformed shape when the element is heated above a particular temperature. Further, the terms recited above can connote, indicate, and/or refer to superelasticity characteristics of a referenced material/element, wherein such shape-memory and/or superelasticity characteristics can relate to the tendency and/or ability of the subject material/element to deform when an external force is applied and return to the undeformed shape when the force is removed; as used herein, a material/element that includes shape memory can be understood to refer the shape memory effect and/or super elasticity. For example, a material/element that includes shape memory properties/characteristics (also referred to as “a shape memory element”) is configured to undergo deformation due to an external force/stress and return to its undeformed shape upon removal of the external force/stress, in some cases by changing the temperature of the material/element, and in other cases without changing the temperature of the material/element. To illustrate, a device with shape memory can include a biased/default shape/form, wherein the device is configured to be compressed, expanded, or otherwise deform when an external force is applied and configured to return to the biased/default shape/form when the external force is removed.

In examples, the shape of the compliant structure 204 can change during expansion and contraction to facilitate a change in volume of a conduit through the implant device 200. For instance, in a state with relatively low luminal pressure (e.g., during diastole), a cross section of the compliant structure 204 can have a first shape that has a relatively small cross-sectional area. In contrast, in a state with relatively high luminal pressure (e.g., during systole), a cross section of the compliant structure 204 can have a second shape that has a relatively large cross-sectional area. One example change in cross-sectional shape is shown below in FIGS. 3-1 and 3-2, wherein the compliant structure 204 changes from an oval to a circle. In examples, the compliant structure 204 expands radially; however, the compliant structure 204 can expand longitudinally or otherwise.

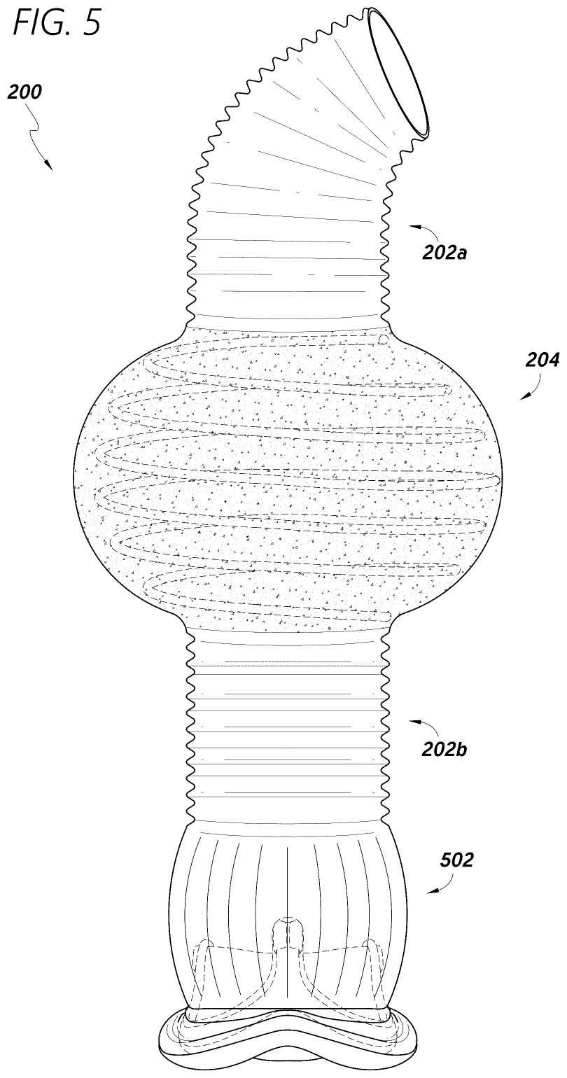

The implant device 200 can be shaped/configured to conform to the shape/form of the associated anatomy in which the implant device 200 is implanted. For example, as shown in FIGS. 2A, 2B, and 2D, the implant device 200 can form a relatively straight structure with respect to a longitudinal axis of the implant device 200 for implantation at a portion of a fluid vessel that is relatively straight, such as the descending thoracic aorta, abdominal aorta, etc. In another example, as shown in FIG. 5, the implant device 200 can at least partially curve relative to a longitudinal axis of the implant device 200 for implantation at a site that has some curve, such as the ascending aorta, aortic arch, etc. In some cases, the implant device 200 is configured to bend to conform to a desired shape. The implant device 200 can include a structure with a lumen to provide a path for blood to flow through the implant device 200, wherein such path can be straight or curved.

The implant device 200 can be implanted at a target site of a blood vessel to treat an aneurysm or other condition and/or to otherwise enhance compliant characteristics of the blood vessel. In some cases, the implant device 200 is implanted to replace a section of a blood vessel that has been resected/cut. To illustrate, the implant device 200 can be implemented as a graft configured to replace an aneurysmal portion of the aorta that has been resected/cut and/or removed. In other cases, the implant device 200 is implanted within a blood vessel while maintaining the current tissue of the blood vessel, even if such tissue is in an undesirable state. To illustrate, the implant device 200 can be implemented as a stent at an aneurysmal site without resecting/cutting the aneurysm, wherein the implant device 200 can expand and contract within the space of the aneurysm. In yet other cases, the implant device 200 is implanted at other places and/or in other contexts.

In examples, the term graft is used in its broad and ordinary meaning and can refer to a device that is used to replace a portion of native tissue that is removed, cut, etc. Although a graft is generally implanted through a surgical procedure, a graft can be implanted through an endovascular procedure (e.g., with a catheter/delivery system). Further, in examples, the term stent is used in its broad and ordinary meaning and can refer to a device that includes a frame-like structure and/or is configured to support tissue in a native state. A stent can generally be configured to compress for delivery to a target site and expand to couple/anchor the device to native tissue. Although a stent is generally implanted though an endovascular procedure, a stent can be implanted in a surgical procedure. In some cases, a graft includes a characteristic(s) of a stent and/or a stent includes a characteristic(s) of a graft. As discussed herein, the implant device 200 can include properties/characteristics of a graft, a stent, and/or other structures. For instance, the implant device 200 can be implanted through a surgical approach and/or an endovascular approach. In examples, the implant device 200 is implemented as a stent-graft that includes a least one characteristic of a stent and a graft.

In some implementations, the implant device 200 can provide relatively large volume changes, even in comparison to a native fluid vessel in a healthy state. For example, in a non-expanded/contracted/default state, the compliant structure 204 can have some amount of concavity such that the diameter, major axis, or minor axis of the compliant structure 204 is smaller than the outer/inner diameter of the native blood vessel. Further, in an expanded state, the diameter, major axis, or minor axis of the compliant structure 204 can be larger than the outer/inner diameter of the native blood vessel, in some cases by more than a threshold amount. One or more of these features can provide relatively large volume changes, in comparison to a blood vessel and/or device that is implanted within a blood vessel and restricted by the walls of the blood vessel.

In examples, the implant device 200 can include one or more anchoring features to anchor/secure/attach the implant device 200 to the native tissue and cause the native tissue to expand and contract with the implant device 200. For instance, the implant device 200 can be configured to be implanted within a native vessel, wherein the implant device 200 can expand and cause one or more anchoring features to couple to the native tissue (e.g., inner tissue wall). Here. the native tissue can conform to the shape/form of implant device 200, such that the native tissue can expand and contract with the expansion and contraction of the implant device 200. In some instances, an anchoring feature includes a barb, patch, pin, coil, screw, tab, hook, wire, spike, or another tissue anchor means configured to embed in and/or hold to the anatomy.

In examples, the implant device 200 is coupled to native tissue such that the native tissue (whether resected or intact) is repositioned over a portion of the implant device 200. This can provide a barrier to protect the surrounding tissue from contacting the implant device 200 directly. However, such repositioning of the tissue may not occur in some cases.

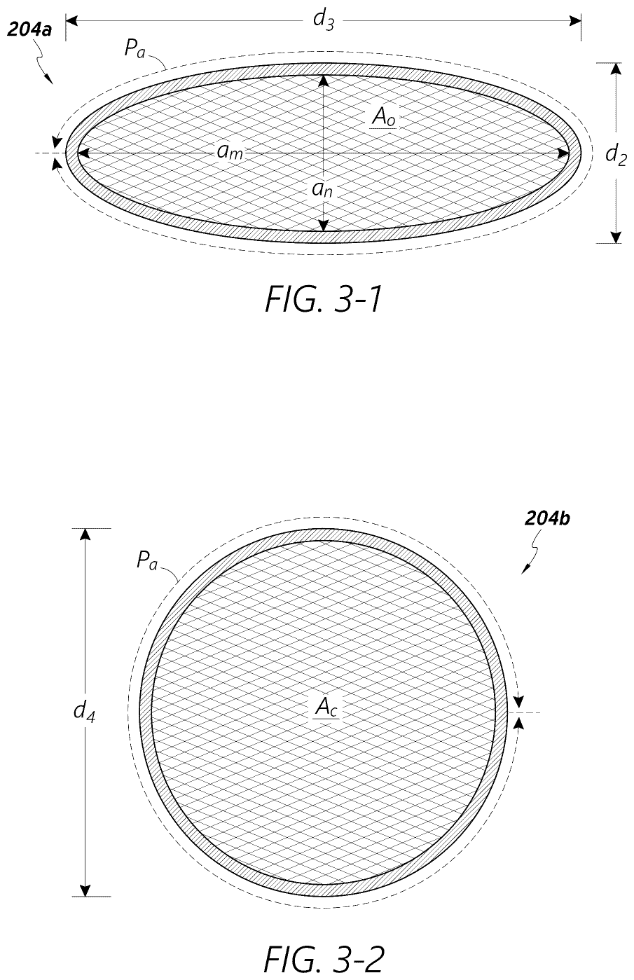

FIGS. 2C-1, 2C-2, and 2C-3 illustrate cross-sectional views of example implementations of the implant device 200 with the compliant structure 204 having different dimensions. In these figures, the compliant structure 204 is illustrated in a non-expanded/default form. FIG. 2C-1 illustrates a cross-sectional view of an example compliant structure 200a with the minor axis aₙ₁ the same length/dimension as or similar to the diameter d₁ of the tube portion 202b (and/or the tube portion 202a, although not shown). Here, the major axis aₘ₁ is larger than the diameter d₁ of the tube portion 202b (and/or the tube portion 202a). FIG. 2C-2 illustrates a cross-sectional view of an example compliant structure 200b with the minor axis aₙ₂ smaller than the diameter d₁ of the tube portion 202b (and/or the tube portion 202a) and the major axis aₘ₂ larger than the diameter d₁ of the tube portion 202b (and/or the tube portion 202a). FIG. 2C-3 illustrates a cross-sectional view of an example compliant structure 200c with both the minor axis aₙ₃ and the major axis aₘ₃ larger than the diameter d₁ of the tube portion 202b (and/or the tube portion 202a).

As discussed above, the compliant structure 204 can expand and contract to change in cross-sectional area and facilitate a change in volume for the implant device 200. In examples, a cross-sectional shape of the compliant structure 204 can change to provide such volume change. FIGS. 3-1 and 3-2 illustrate one example of a change in the cross-sectional area of the compliant structure 204, wherein the compliant structure 204 changes from a non-expanded form having an oval shape (as shown in FIG. 3-1) (e.g., biased non-circular shape) to an expanded form having a circle shape (as shown in FIG. 3-2) as luminal pressure increases within the compliant structure 204. By changing from an oval to a circle, the compliant structure 204 can expand/increase in at least one dimension (e.g., radially along a minor axis), while contracting/decreasing along the major axis. Further, by changing from a circle to an oval, the compliant structure 204 can contract/decrease in at least one dimension (e.g., radially along a minor axis). For case of illustration, the tube portions 202 are not shown in FIGS. 3-1 and 3-2. The cross-sectional shapes are generally illustrated with respect to the line labeled “FIG. 2C” in FIG. 2A.

As shown in FIG. 3-1, when relatively small luminal pressure exists, the compliant structure 204 can have a cross-sectional shape that resembles an oval/ellipse having the major axis aₘ and the minor axis aₙ, which produces the cross-sectional area Aₒ. Here, the state is represented as the compliant structure 204a. As the luminal pressure increases, the compliant structure 204 changes such that the minor axis aₙ increases and the major axis aₘ decreases to provide a cross-sectional shape that generally resembles a circle/circular shape, as represented with the compliant structure 204b in FIG. 3-2. Here, the compliant structure 204b includes area A_{c} that is maximized for the given perimeter/wall-length Pₐ. The area A_{c} is larger than the area Aₒ. In the circular configuration, the diameter d₄ is substantially constant at every angle about the axis of the compliant structure 204. Diverging from this circular cross-sectional shape (e.g., as luminal pressure decreases) can produce a cross-sectional area/volume that is less than the maximum area A_{c} shown in FIG. 3-2. Thus, the compliant structure 204 can typically have the smaller cross-sectional area Aₒ during a lower-pressure period (e.g., diastole) and expand to a form a larger cross-sectional area A_{c} during a higher-pressure period (e.g., systole).

Due to the area Aₒ of the oval configuration shown in FIG. 3-1 being less than the area A_{c} of the circular configuration shown in FIG. 3-2, transitioning from the non-circular shape to the circular shape can provide an increase in area/volume of the implant device 200. Further, the transition back from the circular shape to the non-circular shape can provide a reduction in area/volume of the implant device 200. Such changes can provide compliance characteristics. For example, the implant device 200 can transition between non-circular and circular shapes in response to the typical changes in pressure experienced during the cardiac cycle, which can introduce volumetric change in the implant device 200, thereby increasing cardiac efficiency and/or reducing pulsatile load.

Although various examples are discussed in the context of the shape of the compliant structure 204 changing from an oval to a circle and back to a circle, the compliant structure 204 can alternatively, or additionally, include other characteristics to facilitate a change the area/volume of the implant device 200. For example, the compliant structure 204 can include other shapes, wherein the shape can change or remain the same during expansion and contraction.

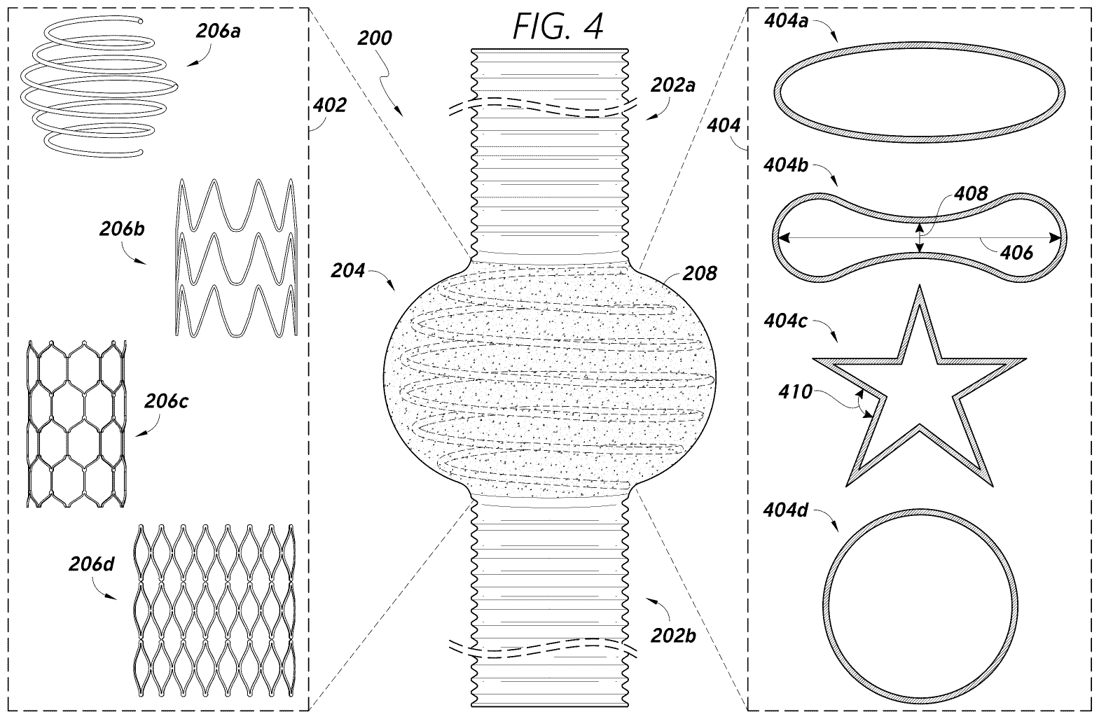

FIG. 4 illustrates various frame types (at 402) that can be implemented for the frame 206 and cross-sectional shapes (at 404) that can be implemented for the compliant structure 204. The cross-sectional shapes are generally illustrated with respect to the line labeled “FIG. 2C” in FIG. 2A. The cross-sectional shapes can be implemented by the frame 206 and/or the covering 208 disposed on the frame 206 (e.g., the cross-sectional shape can include a portion of the frame 206 and/or the covering 208). The cross-sectional shapes generally illustrate default/non-expanded shapes of the compliant structure 204 (e.g., without internal pressure applied to the compliant structure 204). However, the cross-sectional shapes can be implemented in an expanded state.

The frame 206 (also referred to as “the stent or stent frame 206” or “the wire frame 206”) can be made of any at least partially rigid material, such as metal, plastic, etc. For example, the frame 206 can comprise stainless steel, nitinol, etc. Where nitinol or other shape-memory metal or material is implemented, the frame 206 can be biased towards a particular shape. In some cases, the frame 206 is biased towards a default shape having a smaller cross-sectional shape, in comparison to a secondary shape. However, the frame 206 can be biased towards a shape having a larger/maximum cross-sectional shape. The frame 206 can be formed using any suitable process, such as by stamping or machining the frame structure from a sheet or tube of metal.

As shown at 402, the frame 206 can be implemented with a helical/spiral frame shape 206a (e.g., coil), frame sections 206b that are more horizontally disposed (which can include frame sections that are independent from each other and coupled via a covering and/or frame sections that are coupled together at some locations), and/or frames 206c, 206d that have a plurality of cells. The frames 206c and/or 206d can have a structure comprising a plurality of struts forming an array of cells, which can have any suitable or desirable shape (e.g., oval/ellipse, diamond/rhombus, hexagonal diamond/polygon, etc.). The cells can be arranged in any number of columns in the circumferential direction and rows in the axial, or lengthwise, direction. In some implementations, the array of struts is formed from a sheet of metal, which is rolled into a cylinder to form a tubular/cylindrical form. The frame 206 can be formed using any suitable process, such as by stamping or machining the frame structure from a sheet or tube of metal.

Further, as shown at 404, the cross-section of the compliant structure 204 can take a variety of forms/shapes, such as an oval/ellipse 404a, peanut shape 404b, star shape 404c (with any number of points), circle shape 404d, and so on. The oval 404a can include a major axis and a minor axis, wherein the major axis is larger than the minor axis in at least one state of the compliant structure 204, as discussed above. The oval 404a can be configured to expand to a circle, as discussed above in reference to FIGS. 3-1 and 3-2 and elsewhere.

The compliant structure 204 with the peanut shape 404b can include first and second arched end walls/surfaces on opposite ends of a major axis 406 (i.e., opposite major-axis ends). The compliant structure 204 in the peanut shape 404b can also include first and second sidewalls/surfaces on opposite ends of a minor axis 408 (i.e., minor-axis ends) that are deflected radially inward to form a peanut cross-sectional shape, wherein the compliant structure 204/first and second sidewalls are configured to expand to form an oval and eventually a circle (based on luminal/radial pressure within the compliant structure 204). That is, as the luminal pressure increases, the peanut shape 404b can transition to the oval shape 404a, and then, in some cases, transition to the circle shape 404d. This can facilitate a change in cross-sectional area, wherein the peanut shape 404b has the least area given the same perimeter for the shapes. In examples, the peanut shape 404b can provide concavity to produce increased volume change, in comparison to other shapes.

The compliant structure 204 with the star shape 404c can include any number of points. The angle of adjacent edges of each point and/or the dimension of the edges can be designed to facilitate various cross-sectional areas. The compliant structure 204 with the star shape 404c can generally be configured to expand, based on luminal pressure, by reducing an angle between adjacent edges, such as edges 410. This can cause the cross-sectional area to increase. The star shape 404c can transition to another shape during expansion and/or ultimately transition to a circle, in some cases.

The compliant structure 204 with the circular shape 404d can include a diameter that is the same as, larger than, or smaller than a diameter of the tube portions 202 and/or the fluid vessel for which the implant device 200 is implanted. Here, the compliant structure 204 can generally be configured to expand, based on luminal pressure, by increasing the diameter of the compliant structure 204 (e.g., uniformly), such that in an expanded state the compliant structure 204 includes a larger diameter.

FIG. 5 illustrates the implant device 200 in an example with a prosthetic valve 502 attached thereto. In particular, the prosthetic valve 502 can be coupled/attached to the tube portion 202b. The prosthetic valve 502 can be configured to be implanted at a native valve to replace native valve function, while the compliant structure 204 can provide compliance enhancing functions to the native vessel. In one example, the implant device 200 of FIG. 5 is implanted with the prosthetic valve 502 positioned at the aortic valve and the rest of the device 200 extending into the ascending aorta and/or aortic arch. As shown, the tube portion 202a can be at least somewhat curved to fit to the ascending aorta. However, other portions of the implant device 200 can be curved or the tube portion 202a can be straight depending on the implantation context. The prosthetic valve 502 can include one or more leaflets, an annulus feature, and/or other features that mimic a native valve. In examples, one or more ends of the implant device 200 (e.g., tube portions) can be tapered.

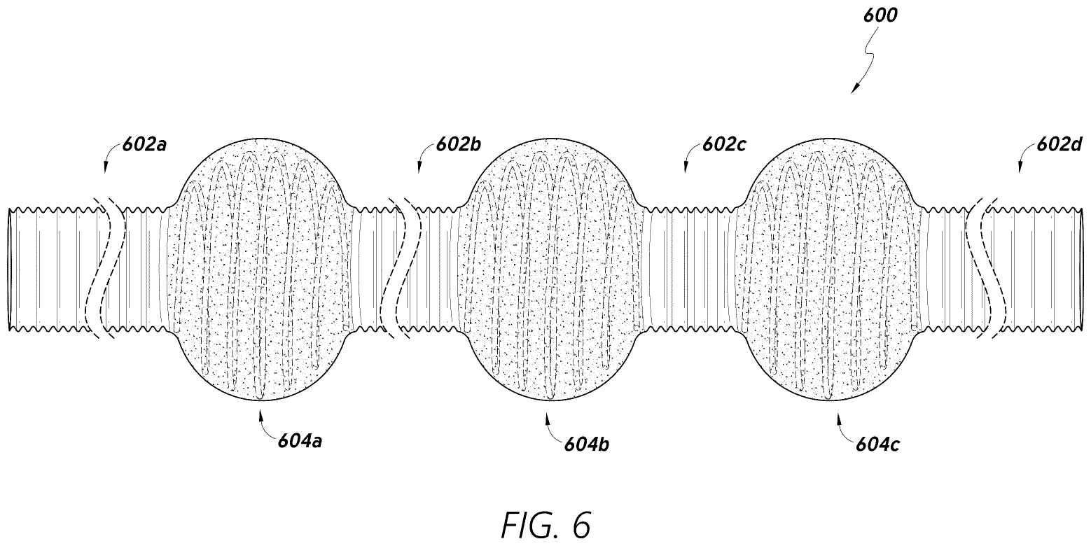

FIG. 6 illustrates an example implant device 600 that includes multiple tube portions 602a-602d coupled to multiple compliant structures 604a-604c. One or more of the compliant structures 604 can correspond to or otherwise include the same/similar features as the compliant structure 204 and/or one or more of the tube portions 602 can correspond to or otherwise include the same/similar features as the tube portions 202. Although a particular number of tube portions 602 and compliant structures 604 are illustrated, any number of tube portions 602 and/or compliant structures 604 can be implemented.

One or more of the compliant structures 604 are configured to be separated/segmented from each other at the tube portions 602, so that the implant device 600 can be customized/configured to a particular size/length. For example, a physician or other user can measure an aneurysmal portion of the aorta to determine a size/length to use for treating/repairing the aorta. If the aneurysmal portion is roughly, for example, the size of two compliant structures 604 (and the associated tube portions), the implant device 600 can be cut at the tube portion 602c so that a length of the implant device 600 is reduced to include the compliant structures 604a and 604b. This can allow the portion of the implant device 600 that is implanted to fit to the aneurysmal portion of the tissue. In examples, the tube portions 602 are formed of a material that is cuttable and/or minimizes fraying (in comparison to other materials), which can allow the implant device 600 to be customized with ease. In some cases, the implant device 600 is of particular use for thoracic and/or abdominal aortic aneurysms, wherein the size of the aneurysm may vary.

In examples, the tube portions 602 of the implant device 600 allow additional tube portions to be connected thereto, such as branch tubes that may project/extend radially therefrom. Further, the tube portions 602 can enable the implant device 600 to flex/curve to conform to a desired shape. For instance, in some examples, the compliant structures 604 can be relatively rigid or inflexible in a longitudinal direction, in comparison to the tube portions 602, wherein the tube portions 602 can flex/bend to allow the implant device 600 to flex/curve in one or more directions and conform to a desired shape, such as a shape of the native tissue for which the implant device 600 is implanted.

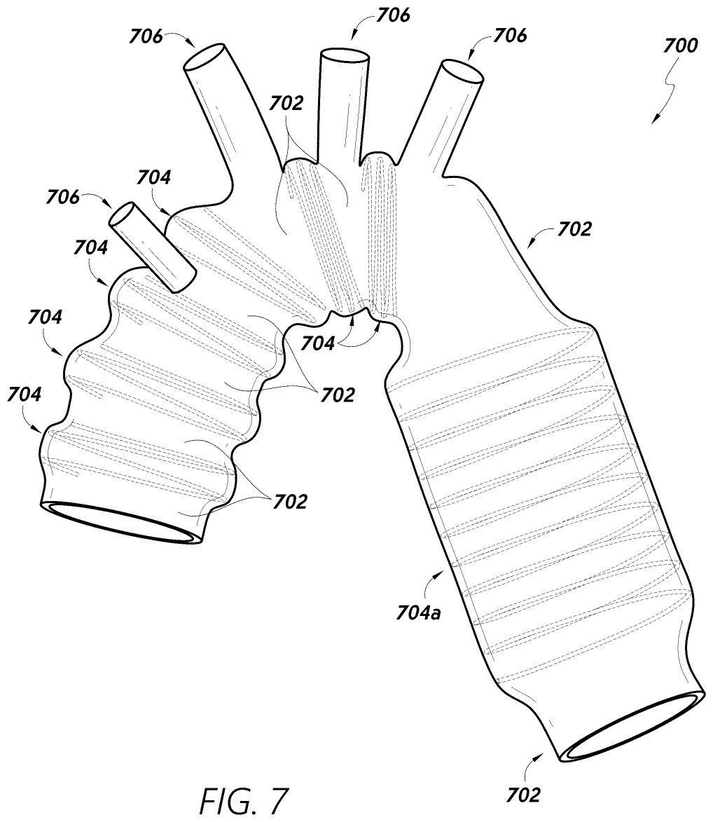

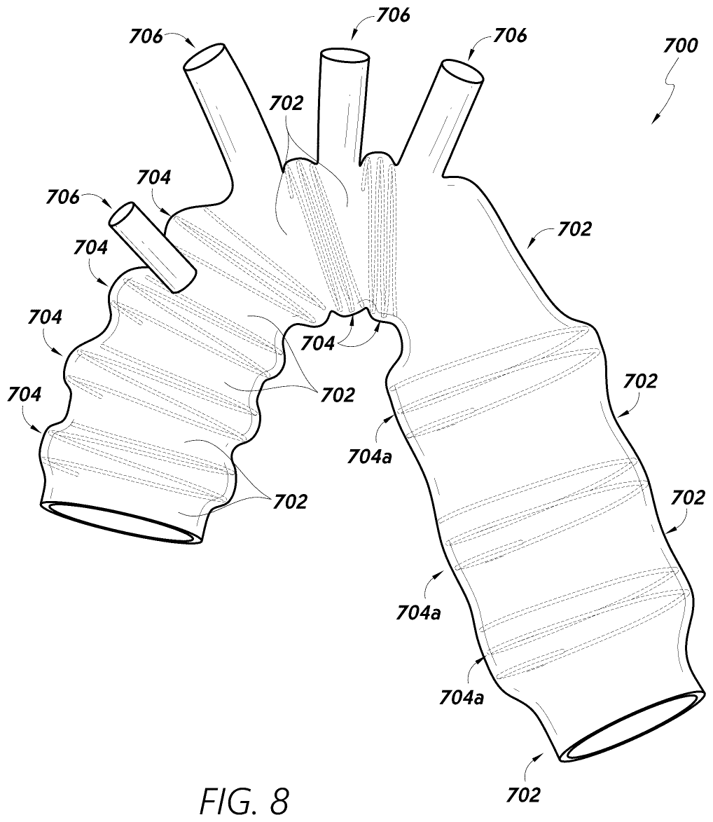

FIGS. 7 and 8 illustrate an example implant device 700 including one or more branch tubes/tube portions/features 706 that radially-project/extend off a main tubular portion of the implant device 700. The main tubular portion can include one or more tubes/tube portions 702 coupled to one or more compliant structures 704. As shown, at least a section of the main tubular portion can be curved (e.g., preformed into a curved/flexed shape along a longitudinal direction), which can provide a desired shape for the implant device 700 that closely matches a shape of a native vessel. One or more of the tube portions 702 can correspond to or otherwise include the same/similar features as the tube portions 202 and/or one or more of the compliant structures 704 can correspond to or otherwise include the same/similar features as the compliant structure 204. Although a particular number of tube portions 702 and compliant structures 704 are illustrated, any number of tube portions 702 and/or compliant structures 704 can be implemented.

FIG. 7 illustrates the implant device 700 with the compliant structure 704a implemented with a substantially uniform component (i.e., a singular form). Whereas FIG. 8 illustrates the implant device 700 with the compliant structure 704a separated into multiple compliant structures/sections/segments to provide additional flexibility/mobility on that side of the device 700 due to the tube portions 702 connected therebetween.

The one or more branch tubes 706 can be fluidly connected to the main tubular portion of the implant device 700. The one or more branch tubes 706 can be integral with the main tubular portion (e.g., the tube portions 702) or separate components that are connected to the main tubular portion. One or more of the branch tubes 706 can be implemented with the same or similar material/structure as the tube portions 702. In examples, one or more of the branch tubes 706 include a smaller diameter (or larger diameter, in some cases) than a diameter of the main tube portion of the implant device 700, such as a diameter of one or more of the tube portions 702 and/or the compliant structures 704 in an expanded or contracted form. In some cases, the branch tubes 706 are frameless, while in other cases the branch tubes 706 include a frame.

In examples, the implant device 700 is designed to replace a native fluid vessel that includes branch vessels. The branch tubes 706 can be configured to match the shape/dimension and/or location of the native branch vessels (e.g., relative to the main native vessel). For instance, the implant device 700 can be designed to replace the aortic arch with the branch tubes 706 dimensioned/shaped to the dimensions/shape of supra-aortic vessels or other vessels, such as a coronary artery, brachiocephalic artery, common carotid artery, subclavian artery, branch for cardiopulmonary perfusion, etc. However, the implant device 700 can be implemented/designed to replace other native vessels. Further, in examples, the implant device 700 can be cut at one or more of the tube portions 702 and/or one or more of the branch tubes 706 to configure the implant device 700 for a particular application/anatomy. For instance, one or more of the tube portions 702 and/or the branch tubes 706 can be cut to the same/similar length as a resected/cut portion of a native vessel.

In examples, the compliant structures 704 is configured to flex/curve/move longitudinally at the tube portions 702 and/or expand/contract radially at the compliant structures 704. This can allow the implant device 700 to flex, expand, and contract as the natural circulation of blood/fluid flows therethrough. In some cases, the implant device 700 is preformed to a shape/curve, such as that shown in FIGS. 7 and 8, while in other cases the tube portions 702 allow the implant device 700 to flex/curve to such shape upon attachment to the associated anatomy.

In examples, the implant device 700 includes a prosthetic valve configured to replace a native valve, similar to or the same as the prosthetic valve 502. The prosthetic valve can be coupled to either end of the implant device 700, depending on the specific application. For instance, the implant device 700 can be configured/designed to replace the aortic arch and aortic valve, wherein a prosthetic valve is attached to the left-hand side of the implant device 700 as shown in FIGS. 7 and 8. The prosthetic valve can be implanted at the native aortic valve with the rest of the implant device extending therefrom and the right-hand side of the implant device 700 attached to a portion of the descending or thoracic aorta, such as a resected portion.

FIGS. 9-1 through 9-3 illustrates an example device/system 900 (sometimes referred to as the “implant device 900”) configured to seal within a fluid vessel to provide compliant characteristics to the fluid vessel. The implant device 900 can generally include a tubular form that provides a fluid/blood channel/conduit to replace/substitute/support a native channel through the fluid vessel. For instance, the implant device 900 can be sealed within a blood vessel at both ends of an aneurysmal site and configured to expand and contract to within a space provided by the aneurysm. In examples, the implant device 900 is implanted through an endovascular approach, wherein the implant device 900 is disposed within a native vessel (e.g., without resecting the native vessel). However, the implant device 900 can be implant through other procedures, such as a more invasive surgical approach.

The implant device 900 can include tubes/tube portions 902a, 902b (also referred to as “the tubular/luminal structures/features/elements 902”) coupled/attached or integral with a compliant structure 904 (also referred to as “the expandable/contractible/elastic structure/feature/component 904”). The implant device 900 can include one or more sealing/anchor/anchoring features/components/structures 910 configured to contact/attach/anchor/seal to an internal portion of a fluid vessel. As shown, the sealing feature 910a is attached to or integral with the tube portion 902a at a first end of the implant device 900 and the sealing feature 910b is attached to or integral with the tube portion 902b at a second end of the implant device 900. Although sealing portions 910 are illustrated, these elements can be eliminated and/or combined with the tube portions 902. For example, the tube portions 902 can alternatively, or additionally, implement/include the functionality/features of the sealing features 910 discussed herein to contact/attach/anchor/seal to an internal portion of a fluid vessel. The tube portions 902a, 902b can be the same as or similar to the tube portions 202a, 202b of the implant device 200. Further, the compliant structure 904 can be the same as or similar to the compliant structure 204 of the implant device 200.

In some examples, the implant device 200 and the implant device 900 are substantially similar and include one or more different characteristics/features that are tailored/designed for different delivery approaches. For instance, the implant device 900 is designed/configured for delivery/implantation through an endovascular approach, whereas the implant device 200 is designed/configured for delivery/implantation through a surgical approach. In some cases, the implant device 900 can include the sealing features 910 and/or a particular amount of oversizing (e.g., by a particular percentage, value, etc.) to facilitate attachment to the internal surface of the anatomy. To illustrate, the tube portions 902, sealing features 910, and/or other elements of the implant device 900 can include a larger dimension relative to the anatomy in which the implant device 900 will be implanted to apply a force and anchor the implant device 900 to the fluid vessel (e.g., a diameter (or length) of the tube portions 902/sealing features 910 in an at least partially expanded or fully expanded state can be larger than an internal diameter of the fluid vessel). Additionally, or alternatively, one or more dimensions of the implant device 900 can be larger (or smaller, in some cases) than the corresponding dimension(s) of the implant device 200. Although the implant devices 200 and 900 are discussed in the context of particular delivery approaches in many examples, the implant devices 200 and 900 can be implanted/delivered in other manners.

The compliant structure 904 (and/or other portions of the implant device 900) can be configured to expand and/or contract radially based on luminal/radial pressure within the fluid vessel to provide a change in volume, in a same/similar manner as that discussed above with reference to the compliant structure 204 of the implant device 200. The compliant structure 904 can include a frame 906 and/or a covering/cover 908 disposed on the frame 906. The covering 908 can be disposed around and/or within the inside of the frame 906, such that the covering 908 generally contacts and conforms to the shape/form of the frame 906 (e.g., expands and contracts along with the frame 906). In implementations, the covering 908 (and/or the covering 208) comprises a cloth or polymer sleeve which may be at least partially clastic, or alternatively, nonelastic. The covering 908 can be applied over or within the frame in any suitable or desirable manner. For example, the covering 908 can be applied using an electrical or mechanical spinning (e.g., rotary jet spinning. electrospinning, or similar) application process or other deposition process. The covering 908 can be coupled to the tube portions 902 to form a fluid tight seal with the tube portions 902 and provide a conduit for fluid flow through the implant device 900. The frame 906 can be the same as or similar to the frame 206 of the implant device 200 and/or the covering 908 can be the same as or similar to the covering 208 of the implant device 200.

The compliant structure 904 can be biased to a particular shape/form (also referred to as “a biased/contracted state/form”), wherein such biased shape/form is associated with less volume/cross-sectional area than a non-biased form. As luminal pressure within the implant device 900 increases, the compliant structure 904 can expand to the non-biased shape/form (also referred to as “an expanded state/form” or “secondary state/form”). The frame 906 can include shape memory/super elasticity to implement the biased form/shape. For instance, the frame 906 can be formed of nitinol or other shape-memory metal or material, which can allow the frame 906 to expand and return to the biased form. Although various figures show the implant device 900 with the frame 906, the implant device 900 can be implanted without a frame, such as by using just an elastic/flexible tube or another structure that facilitates expansion and contraction.

The sealing features 910 and/or the tube portions 902 can be configured to couple/attach/contact a native fluid vessel (not illustrated) and/or seal the implant device 900 to the native fluid vessel. For example, the sealing features 910 and/or the tube portions 902 can be configured to fluidly seal the implant device 900 to an internal surface of a blood vessel (in the case of implementing an endovascular procedure) to cause blood to flow through a lumen in the implant device 900 and avoid/minimize blood flow around the implant device 900. In some cases, the sealing features 910 and/or the tube portions 902 are implemented to provide a sealed path for blood to flow through the implant device 900 and prevent blood from collecting around the implant device 900 between the implant device 900 and the native vessel, which can lead to complications. Although the implant device 900 is illustrated with a sealing feature at each end of the implant device 900, the implant device 900 can include any number of sealing features.

The sealing features 910 and/or the tube portions 902 can generally include a cross-sectional shape that matches or is otherwise similar to a cross-sectional shape of the fluid vessel in which the implant device 900 is implanted. For example, in the case of implanting the device 900 in the aorta, the sealing features 910/tube portions 902 can include circle/circular cross-sectional shapes such that the sealing features 910/tube portions 902 can fit to the cross-sectional shape of the aorta to form a tight seal therewith. However, the sealing features 910/tube portions 902 can take any form/shape. In contrast, the compliant structure 904 can include a non-circular cross-sectional shape (in a non-expanded/default/biased form) or another shape/structure that is configured to be expanded. For instance, the compliant structure 904 can take an oval or peanut cross-sectional shape, as similarly discussed above for the compliant structure 204 of the implant device 200. Further, the compliant structure 904 can have any of the characteristics of the compliant structure 204.

In examples, the compliant structure 904, the sealing features 910, and/or the tube portions 902 can be implemented as a stent/frame structure. In some cases, the frame can have a structure comprising a plurality of struts forming an array of cells, which can have any suitable or desirable shape (e.g., oval/ellipse, diamond/rhombus, hexagonal diamond/polygon, etc.). The cells can be arranged in any number of columns in the circumferential direction and rows in the axial, or lengthwise, direction. In some implementations, an array of struts is formed from a sheet of metal, which is rolled into a cylinder to form the tubular/cylindrical form of the frame configured for placement within a blood vessel. In some implementations, a balloon catheter is used for delivery and/or to expand the frame for securing in the wall of an artery or other blood vessel or body cavity. The frame can be formed using any suitable process, such as by stamping or machining the frame structure from a sheet or tube of metal. The frame can be made of any at least partially rigid material, such as metal, plastic, etc. For example, the frame can comprise stainless steel or nitinol. The frame can be self-expanding or expandable by another device (also referred to as an “expandable frame”) such that the frame can be configured to expand radially from a compressed configuration (e.g., during delivery) to the expanded state.

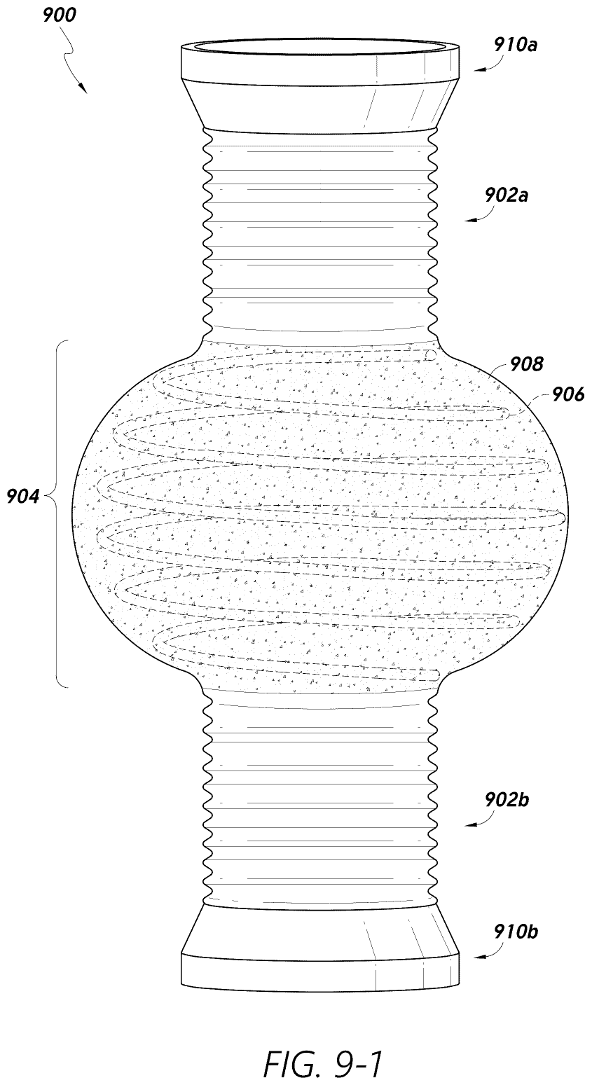

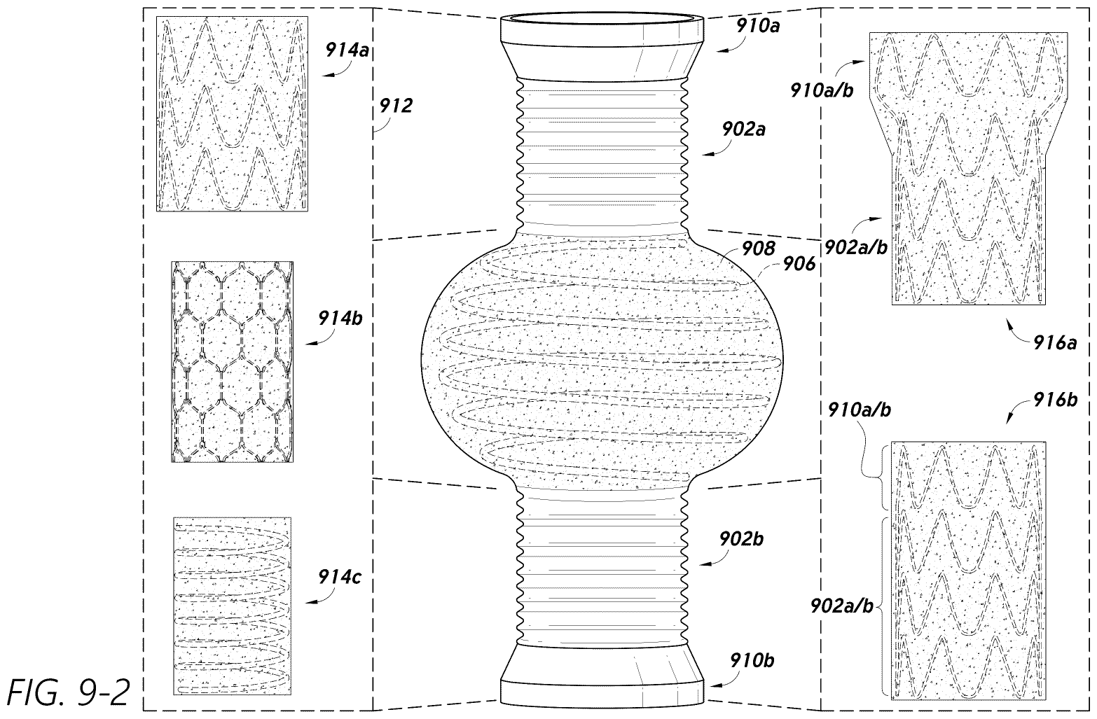

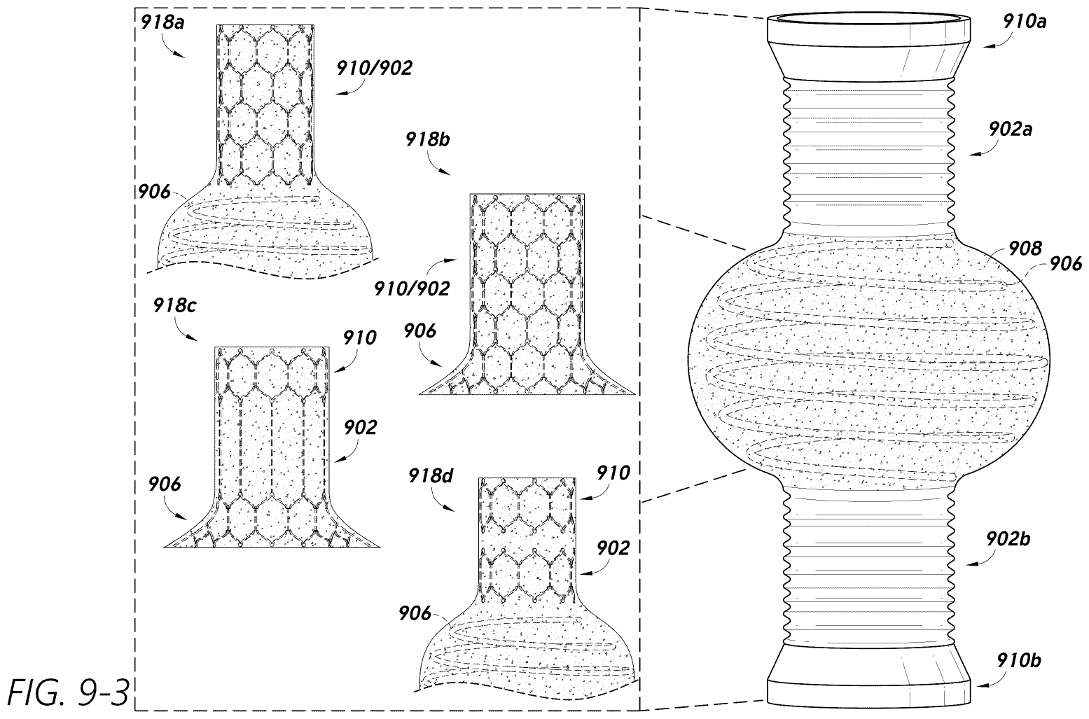

FIG. 9-2 illustrates examples of the sealing features 910 and/or the tube portions 902 implemented with one or more stent/frame structures. As shown at 912, a frame of one or more of the sealing features 910 and/or the tube portions 902 can be implemented with frame sections 914a that are more horizontally disposed (which can include frame sections that are independent from each other and coupled via a covering and/or frame sections that are coupled together at some locations), frames 914b that have a plurality of cells, a frame 914c having helical/spiral frame shape (e.g., coil), and/or other types of frames. The frame 914b can have a structure comprising a plurality of struts forming an array of cells, which can have any suitable or desirable shape (e.g., oval/ellipse, diamond/rhombus, hexagonal diamond/polygon, etc.). The cells can be arranged in any number of columns in the circumferential direction and rows in the axial, or lengthwise, direction. In some implementations, the array of struts is formed from a sheet of metal, which is rolled into a cylinder to form a tubular/cylindrical form. The frames 914 can be formed using any suitable process, such as by stamping or machining the frame structure from a sheet or tube of metal. The frames 914 can be implemented as any of the frames discussed herein, such as a frame discussed for the implant device 200 or any other device discussed herein.

In some examples where the sealing features 910 and/or the tube portions 902 are implemented as a stent/frame(s), the outside or inside of the frame is covered with a fabric, polymer cover, or other material. The covering/cover can promote tissue ingrowth within the inner diameter/surface of the native blood vessel. The cover can be disposed on an outer surface or area of the frame and/or can be disposed/applied to the inner diameter of the frame on an inside thereof. In some implementations, the cover comprises a cloth or polymer sleeve which may be at least partially clastic, or alternatively, nonelastic. The cover can be applied over or within the frame in any suitable or desirable manner. For example, the cover can be applied using an electrical or mechanical spinning (e.g., rotary jet spinning, electrospinning, or similar) application process or other deposition process.

In some examples, the sealing features 910 and/or the tube portions 902 expand radially toward the distal ends (e.g., a diameter/radius of the sealing feature 910a at a distal end relative to the compliant structure 904 is larger than a diameter/radius of the tube portion 902a), as shown at 916a in FIG. 9-2. Further, in some examples, the sealing features 910 and/or the tube portions 902 form a relatively constant cylindrical structure (e.g., a diameter/radius of the sealing feature 910a is the same as or similar to a diameter/radius of the tube portion 902a), as shown at 916b in FIG. 9-2. For ease of illustration/discussion, the sealing features 910 and the tube portions 902 are shown at 916a and 916b in a combined format with a distal end facing upward (e.g., the sealing features 910a and 910b both facing upward), even though the lower sealing feature 910b and tube portion 902b are positioned facing downward in the middle of FIG. 9-2.

Further, in some examples where the sealing features 910 and/or the tube portions 902 are implemented as a stent/frame(s), the frame 906 of the compliant structure 904 and the frame of the sealing feature(s) 910/tube portion(s) 902 are separate frames/components, as shown in the examples at 918a and 918d in FIG. 9-3. Moreover, in examples, the frame 906 of the compliant structure 904 and a frame of the sealing feature(s) 910/tube portions 902 can be integral frames (as shown at 918b in FIG. 9-3). Further, in examples, a frame structure of a sealing feature 910 can be coupled/attached to the frame 906 of the complaint structure 904 via a frame element (e.g., wire, strut, etc.) that runs through one or more of the tube portions 902a, 902b (as shown at 918c in FIG. 9-3). In examples, one or more of these implementations provides a more simplified manufacturing process, in some cases. In examples, one or more frames/frame structures (whether integral, coupled, or separate frame structures/elements) can be coupled together with a covering/cloth. For ease of illustration/discussion, the sealing features 910 and the tube portions 902 are shown at 918 in a combined format with a distal end facing upward, even though the lower sealing feature 910b and tube portion 902b are positioned facing downward in the middle of FIG. 9-3.

In examples, the sealing features 910 and/or the tube portions 902 can be implemented as different types of structures other than a stent/frame. For instance, the sealing features 910/tube portions 902 can each be implemented as a ring/band, such as a non-continuous band having a gap/break/space in the ring that is configured to allow the ring to be compressed for delivery and expanded upon delivery to seal to the surrounding tissue. The band can be made of an at least partially rigid material, such as a metal, plastic, or another material.

Although the sealing features 910 are illustrated in many examples with larger diameters at the ends of the implant device 900, the sealing features 910 can include the same diameters as the tube portions 902. Further, in some examples, the tube portions 902 and sealing features 910 can be implemented together such that a combined tube-sealing feature extends from an end of the implant device 900 to the compliant structure 904, with or without the expansion shown in various figures. For example, a combined tube-sealing feature/component can be configured to contact/attach/anchor/seal to an internal portion of a fluid vessel. A combined tube-sealing feature can include features of the tube portions 902 and/or features of the sealing features 910. Moreover, the sealing features 910 can be eliminated and the functionality of the sealing features 910 can be implemented entirely, or partially, by the tube portions 902.

As used herein, reference to a sealing feature can refer to a specific/separate sealing element and/or a tube portion (when the tube portion is implemented at least in part to seal/anchor/attach to the anatomy).

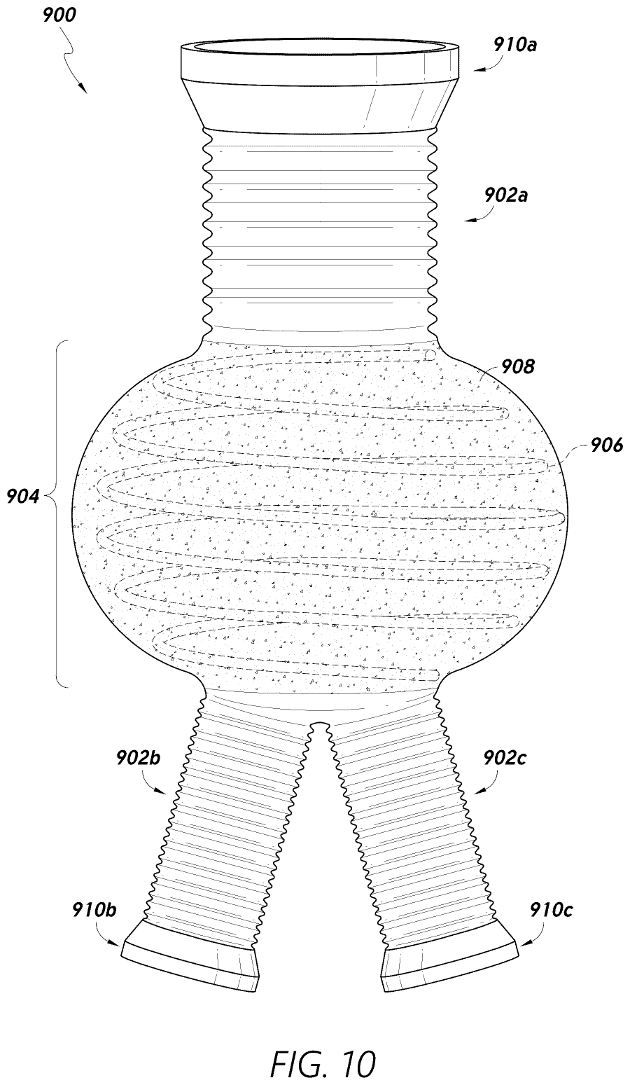

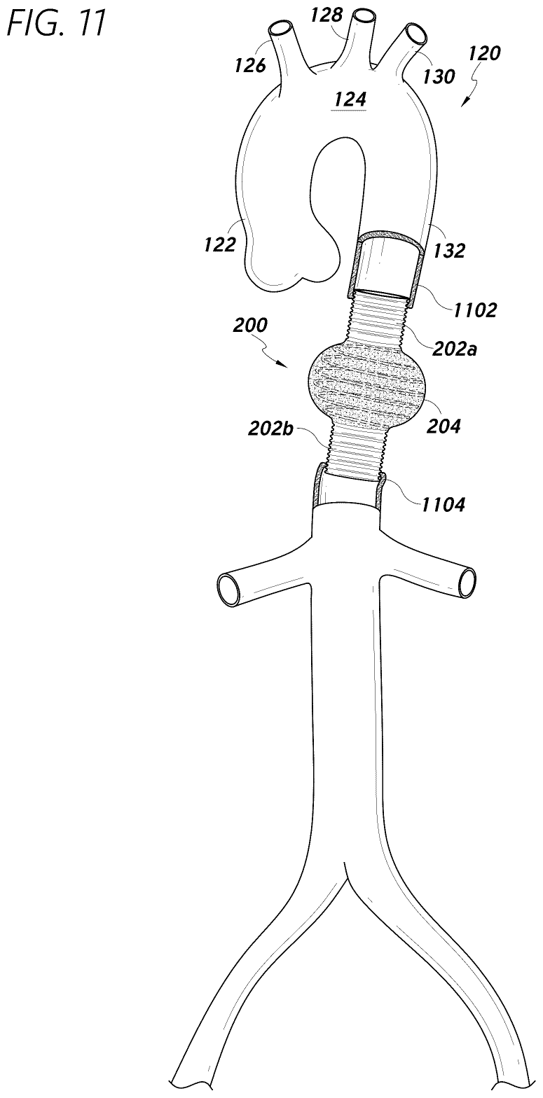

FIG. 10 illustrates the implant device 900 implemented with two lower tubes/tube portions 902b, 902c having sealing features 910b, 910c, respectively. In this implementation, the implant device 900 can be placed at a location having a bifurcation (e.g., a first channel that divides into two or more channels downstream or upstream). As such, the implant device 900 can be referred to as a “bifurcation or branching implant device.”

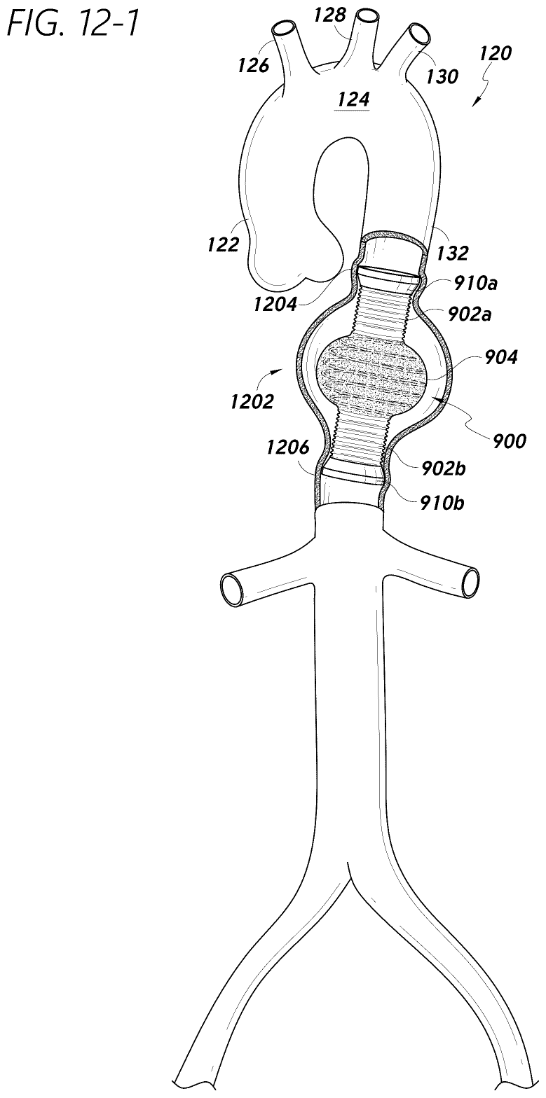

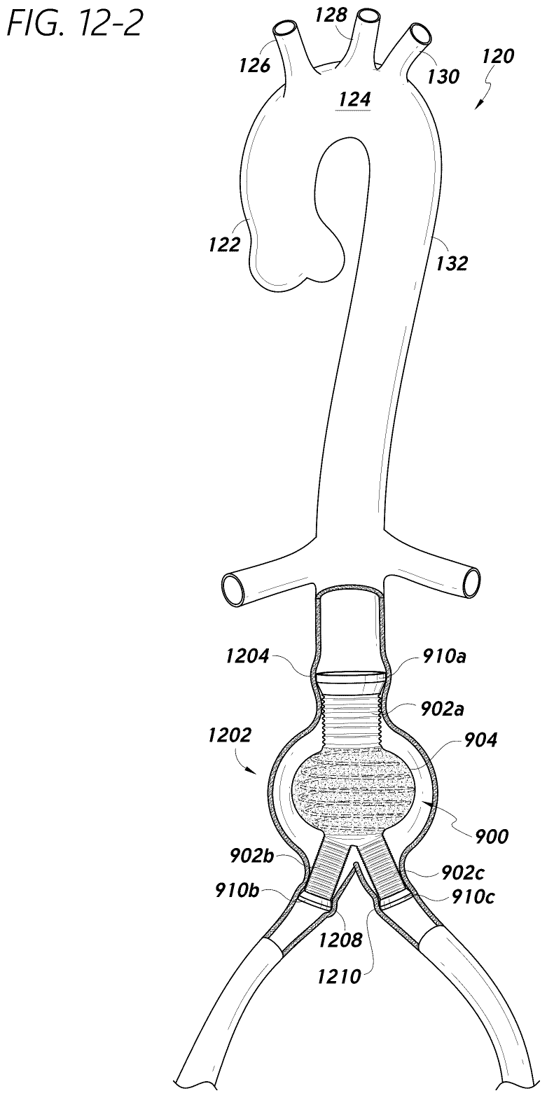

As shown, the compliant structure 904 includes the first tube 902a (having the sealing feature 910a) coupled to a first end of the compliant structure 904, a second tube 902b (having the sealing feature 910b) coupled to a second end of the compliant structure 904, and third tube 902c (having the sealing feature 910c) coupled to the second end of the compliant structure 904. The sealing features 910a, 910b, 910c are disposed at opposite ends of the tubes 902 relative to an attachment end of the tubes 902 to the compliant structure 904. For instance, tube portion 902b is attached to the compliant structure 904 at a first end of the tube 902b, while the sealing feature 910b is integral with or attached to a second end of the tube 902b. In examples, the first tube 902a is configured to be disposed within a fluid vessel, while the second and third tubes 902b, 902c are configured to be disposed within branches of the vessel, as discussed below in reference to an example shown in FIG. 12-2.

In examples, the implant device 900 from FIG. 9 or 10 includes any of the features of the implant device 200 discussed herein. For instance, the implant device 900 can include a prosthetic valve (such as that illustrated in FIG. 5), multiple compliant structures (such as that illustrated in FIG. 6), one or more branch tubes (such as that illustrated in FIGS. 7 and 8), an arch shape (such as that illustrated in FIGS. 7 and 8), and so on.

FIG. 11 illustrates the implant device 200 implanted within example anatomy of the patient, namely a resected portion of the aorta 120. Here, the aorta 120 is resected and the first tube portion 202a is attached to an upper/upstream portion 1102 of the aorta 120 (e.g., a first location) and the second tube portion 202b is attached to a lower/downstream portion 1104 of the aorta 120 (e.g., a second location). The aorta 120 can be resected before, during, or after implantation. In some cases, one or more of the tube portions 202a, 202b are disposed/slid within the aorta 120, as shown in FIG. 11. In other cases, the aorta 120 is disposed within one or more of the tube portions 202a, 202b. The tube portions 202a, 202b can be attached to the aorta 120 in a variety of manners, such as by using sutures, bands, or other anchoring/attachment structures/means that are configured to provide a fluid tight seal between the implant device 200 and the aorta 120. The tube portions 202a, 202b can be similar in diameter and/or cross-sectional shape to the aorta 120 (e.g., circular) so that the tube portions 202a, 202b can fit within or around the resected aorta 120. Meanwhile, the compliant structure 204 can have a different shape in some cases, as discussed herein.

In examples, the implant device 200 is coupled to native tissue such that the native tissue is positioned/repositioned over a portion of the implant device 200. For example, a cut portion of the aorta 120 can be folded over one or more portions of the implant device 200 and/or tissue may grow over the implant device 200 after implantation. This can provide a barrier to protect the surrounding tissue from contacting the implant device 200 directly, such as the compliant structure 204 that expands and contracts. However, the implant device 200 can additionally, or alternatively, include a covering that minimizes tissue friction.

In the example of FIG. 11, the implant device 200 is generally implemented as a graft, wherein an aneurysmal portion of the aorta 120 is resected/removed so that the implant device 200 can replace the aneurysmal portion. The implant device 200 can be implanted through a surgical procedure or another procedure. In this example, the compliant structure 204 is free to expand and contract without constraint from vessel walls, in contrast to some cases where the compliant structure 204 is implanted within the aorta 120. Further, the graft implementation can allow the compliant structure 204 to be designed to provide a relatively large volume change (e.g., more than a threshold, more than a native portion of the aorta of similar length, etc.), thereby maximizing compliance characteristics of the implant device 200. Moreover, the graft implementation can avoid blood or other fluids from collecting within the aorta between the implant device 200 and an inner wall of the aorta 120.

Although the implant device 200 is illustrated as implanted within the aorta 120 in this example, the implant device 200 can be implanted within other anatomy and/or positioned elsewhere within the aorta 120. Further, the implant device 200 can be implanted in anatomy that is not resected.

FIGS. 12-1 and 12-2 illustrates the implant device 900 implanted within example anatomy of the patient, namely an aneurysmal portion/section 1202 of the aorta 120. FIG. 12-1 shows the implant device 900 from FIG. 9 implanted within an aneurysm in the thoracic aorta. Here, the aneurysm is treated with a relatively straight medical device. FIG. 12-2 shows the implant device 900 from FIG. 10 implanted within an aneurysm near the aortic bifurcation. Here, the aneurysm is treated with a branching/bifurcation medical device. However, the implant device 900 from FIGS. 9 and/or 10 can be implanted at other locations within the aorta or other fluid vessels.

In the examples of FIGS. 12-1 and 12-2, the implant device 900 is generally implemented as a stent or stent-grant and/or implanted through an endovascular procedure or another minimally invasive procedure, which can avoid complications associated with more invasive surgical procedures. In such minimally invasive procedures, the aneurysmal portion 1202 of the aorta 120 is generally not resected. However, other types of procedures can be implemented.

As shown in FIGS. 12-1 and 12-2, the implant device 900 is disposed within the aorta 120 to position the compliant structure 904 within a space created by the aneurysmal portion 1202 (i.e., the aneurysmal sac). That is, the implant device 900 is positioned such that the compliant structure 904 is within a dilated/deformed/enlargement portion/space of the aneurysm (i.e., the aneurysmal sac). In such position, the compliant structure 904 is free to expand and contract within the space of the aneurysmal portion 1202, which may generally be larger in space relative to neighboring portions of the associated vessel. In view of this positioning, the compliant structure 904 can be designed to provide a relatively large volume change (e.g., more than a threshold, more than a native portion of the aorta of similar length, etc.), thereby maximizing compliance characteristics of the implant device 900 and/or maximizing the space created by the aneurysm.

In the example of FIG. 12-1, the first sealing feature 910a is sealed to an inner surface/wall of an upper/upstream portion 1204 of the aorta 120 above the aneurysm and the second sealing feature 910b is sealed to an inner surface/wall of a lower/downstream portion 1206 of the aorta 120 below the aneurysm.

Meanwhile, in the example of FIG. 12-2, the first sealing feature 910a is sealed to an inner surface/wall of an upper/upstream portion 1208 of the aorta 120 above the aneurysm, the second sealing feature 910b is sealed to an inner surface/wall of the right common iliac artery 1210 below the aneurysm, and the third sealing feature 910c is sealed to an inner surface/wall of the left common iliac artery 1212 below the aneurysm.

The sealing features 910a, 910b, 910c and/or tube portions 902a, 902b, 902c can be implemented as expandable frames/structures that are self-expandable or device-expandable to radially expand and attach the implant device 900 to ends of the aneurysmal portion 1202. When implanted, the sealing features 910a, 910b, 910c and/or tube portions 902a, 902b, 902c can seal to tissue/anatomy to provide a fluid path through the implant device 900 and avoid blood flow into a space between the implant device 900 and the inner wall of the aorta 120 at the aneurysmal portion 1202 (e.g., avoid blood from collecting in the aneurysmal sac). The sealing features 910a, 910b, 910c and/or tube portions 902a, 902b, 902c can be similar in cross-sectional shape to the aorta 120/iliac artery 1210/1212 so that the sealing features 910a, 910b, 910c and/or tube portions 902a, 902b, 902c can fit within (or around) the associate vessel.

In an expanded/implanted form, the sealing features 910a, 910b, 910c and/or tube portions 902a, 902b, 902c can generally include a diameter/cross-sectional dimension that is larger than the inner diameter/cross-sectional dimension of the associated vessel in which the implant device 900 is implanted. For example, the sealing features 910a. 910b, 910c and/or tube portions 902a, 902b, 902c can each, in an expanded form, have an outer diameter (or inner diameter, in some cases) that is larger than or the same as an average or patient specific inner diameter of a healthy/neighboring blood vessel. In examples, the diameter/cross-sectional dimension of the sealing features 910a, 910b, 910c and/or tube portions 902a, 902b, 902c can be larger than a healthy/neighboring blood vessel by a particular amount (e.g., a particular percentage of oversizing).

Further, in examples, an outer/inner diameter/cross-sectional dimension of the compliant structure 904 in an expanded or non-expanded form can be the same as or larger than (or smaller, in some cases) (i) an inner/outer diameter/cross-sectional dimension of one or more of the tube portions 902a, 902b, 902c (e.g., frame/covering), (ii) an inner/outer diameter/cross-sectional dimension of one or more of the sealing features 910a, 910b, 910c in an expanded or non-expanded form (e.g., a frame of a sealing feature), and/or (iii) an inner/outer diameter/cross-sectional dimension of a healthy/neighboring blood vessel (e.g., a healthy section of the aorta 120 that is within a predetermined proximity to the aneurysm). In examples, the diameter/cross-sectional dimension of the compliant structure 904 can be larger than such feature by a particular amount (e.g., a particular percentage of oversizing).

Although the implant device 900 is illustrated in the examples of FIGS. 12-1 and 12-2 as implanted within the aneurysmal portion 1202, the implant device 900 can be implanted within other anatomy and/or positioned elsewhere. For example, the implant device 900 can be disposed within other dilated tissue/enlargements, such as other anatomy that has been dilated in the same or other ways, other portions of the aorta 120 that have been dilated, etc. Further, the implant device 900 can be implanted in anatomy that is resected.

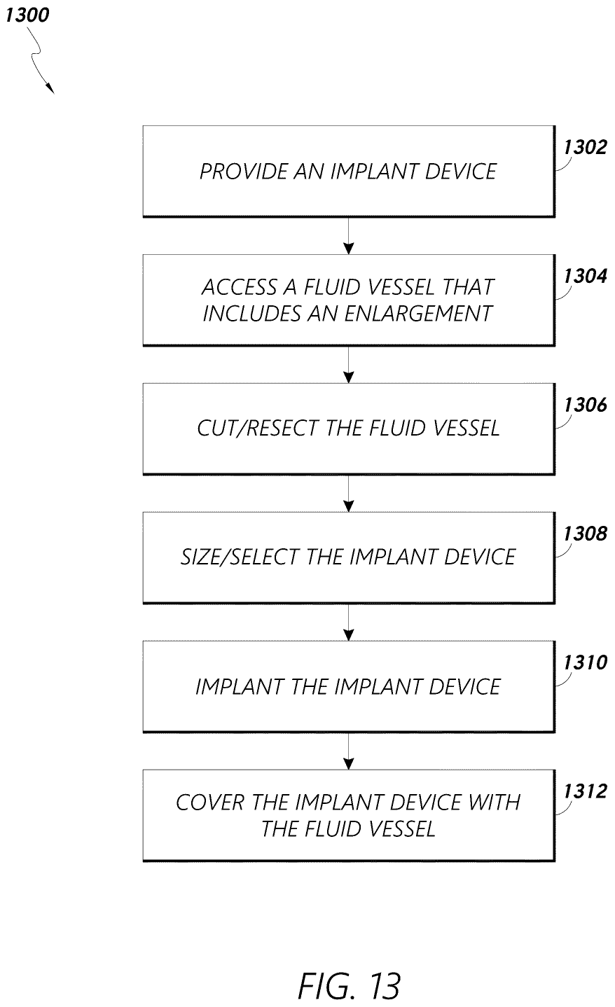

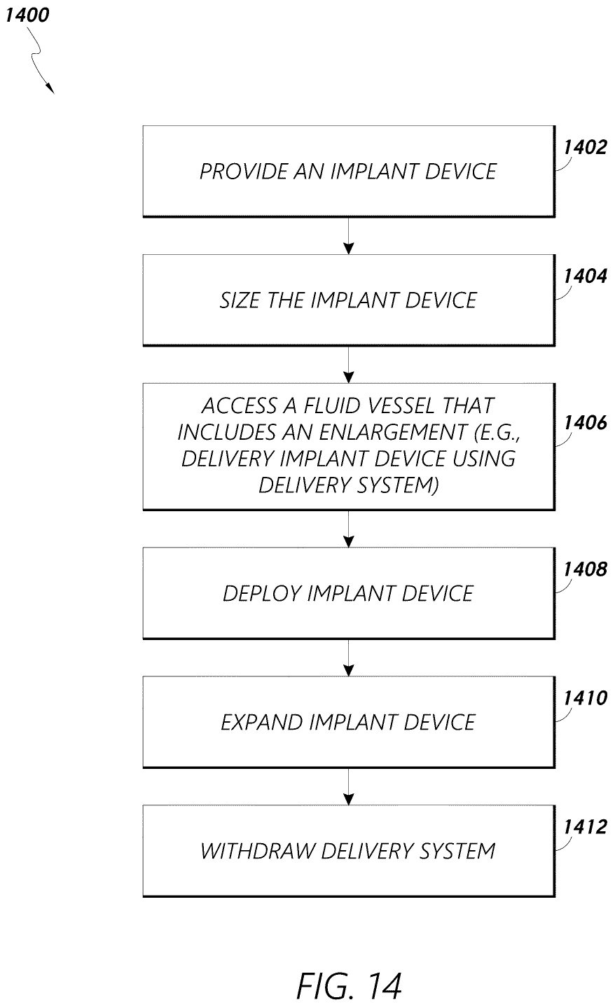

FIGS. 13 and 14 illustrate flow diagrams for process 1300 and 1400, respectively, for implanting an implant device (including any of the implant devices discussed herein) within anatomy in accordance with one or more examples. In examples, the process 1300 of FIG. 13 relates to a more invasive/surgical procedure, wherein the target site is accessed through a surgical approach and/or the target tissue is surgically cut/resected. In contrast, the process 1400 of FIG. 14 relates to an endovascular/minimally invasive procedure, wherein the target site is accessed through a endovascular/minimally invasive approach and/or the target tissue is not cut/resected. However, any of the blocks/acts discussed for the process 1300 and/or the process 1400 can be implemented in the context of a surgical or endovascular/minimally invasive approach. Further, a block/act illustrated for the process 1300 of FIG. 13 can be implemented for the process 1400 of FIG. 14, and vice versa. Although the blocks are illustrated in a particular order, the order of the blocks can be modified. Further, one or more of the blocks can be eliminated from the processes 1300 and/or 1400.

Although various aspects of the processes 1300 and 1400 and certain other examples are described herein in the context of the aorta, the devices of the present disclosure can be implanted in other arterial or venous blood vessels, such as the inferior vena cava. Further, although the processes 1300 and 1400 and accompanying illustrations are presented with respect to the implantation of a single compliance-enhancement implant device, the processes 1300 and 1400 can involve implanting multiple compliance-enhancement implant devices in various positions within the aorta and/or other fluid vessels.

In FIG. 13, at block 1302, the process 1300 includes providing an implant device. The implant device can include any of the implant devices discussed herein, such as the implant device 200, the implant device 600, the implant device 700, the implant device 900, or other implant devices discussed herein. In one non-limiting example, the implant device 200 is implemented as a graft that includes the first tube 202a, the compliant structure 204 coupled to the first tube 202a, and the second tube 202b coupled to the compliant structure 204.

At block 1304, the process 1300 includes accessing a fluid vessel that includes an enlargement. For example, a physician/user or robotics system can surgically open a patient to access an aneurysm/target site in the aorta or another fluid vessel. Alternatively, or additionally, in some cases, a catheter/medical tool is implemented to access the target site through a percutaneous access point/port or natural orifice.

At block 1306, the process 1300 includes cutting and/or resecting the fluid vessel. For example, a physician/user or robotics system can cut/resect an aneurysm in the aorta, which can include removing at least a portion of the aneurysmal tissue. In some cases, a catheter/medical tool is implemented to cut/resect the target tissue.

At block 1308, the process 1300 includes sizing and/or selecting the implant device. For example, an aneurysm/aneurysmal portion of the aorta can be measured using various techniques to determine a size/dimension of the aneurysm/aneurysmal portion, such as a length, width, diameter, or other dimension. This can include measuring a distance between resected portions of the fluid vessel (when performed after block 1306) and/or measuring a length or other dimension of the aneurysm/aneurysmal portion before the aneurysm/aneurysmal portion is resected/cut (when performed before block 1306). In examples, a size/dimension of neighboring health tissue is measured/determined, such as a diameter or other dimension of the aorta within a predetermined distance to the aneurysm/aneurysmal portion.

In examples, an implant device can be cut to a length that satisfies/matches the length of the aneurysm. For instance, one or more tube portions of an implant device (which can be located on the ends of the device) can be cut to create the appropriate longitudinal length for the implant device. Additionally, or alternatively, an implant device can include multiple compliant structures coupled together via multiple tube portions (such as that shown in FIG. 6), wherein the implant device can be cut at one of the tube portions to configure the implant device with the appropriate longitudinal length.

Further, in examples, multiple implant devices are available with different sizes/dimensions so that a physician/user can select the most appropriate implant device for the patient/anatomy. To illustrate, tube portions and/or compliant structures of different implant devices can include different diameters and/or lengths such that a physician/user can select an implant device that satisfies/matches the size/dimension of the aneurysm/aneurysmal portion and/or the size/dimension of neighboring health tissue. For instance, an implant device that has tube portions with the same/similar diameter as the aorta can be selected.

At block 1310, the process 1300 includes implanting the implant device. For example, a physician/user or robotic system can attach a first tube of the implant device (at a first end of the device) to a first location above the enlargement and attach a second tube of the implant device (at a second end of the device) to a second location below the enlargement. The implant device can be attaching using sutures and/or other attachment features/means. The implant device can be implanted before or after the fluid vessel is resected/cut (i.e., before or after block 1306). In some examples, the implant device is implanted at or near a native valve. In cases, where the implant device includes a prosthetic valve, the prosthetic valve can be placed at the native valve.

The term “suture” is used herein according to its broad and ordinary meaning and may refer to any elongate cord, strip, strand, line, rope, wire, filament, tie, string, ribbon, strap, or portion thereof, or other type/form of material used in medical procedures (e.g., ePTFE suture, for example, GORE-TEX® sutures, W.L. Gore, Newark, Delaware). Furthermore, examples of the present disclosure can be implemented in connection with non-surgical and/or non-biological suture/line tensioning. With respect to the present disclosure, one having ordinary skill in the art will understand that a wire or other similar material can be used in place of a suture. Furthermore, in some contexts herein, the terms “cord” and “suture” can be used substantially interchangeably. In addition, use of the singular form of any of the suture-related terms listed above, including the terms “suture” and “cord,” can be used to refer to a single suture/cord, or to a portion thereof. For example, where a suture knot or anchor is deployed on a distal side of a tissue portion, and where two suture portions extend from the knot/anchor on a proximal side of the tissue, either of the suture portions can be referred to as a “suture” or a “cord,” regardless of whether both portions are part of a unitary suture or cord. Anchor guides in accordance with aspects of the present disclosure can be utilized in methods for controlling spacing of surgical sutures. Such sutures and/or associated anchors can be introduced to the target implantation site using a minimally invasive incision and/or can be implanted/deployed while the patient's heart is beating. Furthermore, sutures can be used with a pledget to reduce tissue damage and/or spread the suture load over a broader surface area.

At block 1312, the process 1300 includes covering the implant device with the fluid vessel. For example, a physician/user or robotics system can take a loose piece of the aorta that was resected and is not attached to the implant device and cover at least a portion of the implant device. This can provide a barrier between the implant device and the neighboring tissue of the patient. Although illustrated in FIG. 13, in examples block 1312 is not performed.

In FIG. 14, at block 1402, the process 1400 includes providing an implant device. The implant device can include any of the implant devices discussed herein, such as the implant device 200, the implant device 600, the implant device 700, the implant device 900, or other implant devices discussed herein. In one non-limiting example, the implant device 900 is implemented as a stent or stent-graft that includes the first tube 902a, the compliant structure 904 coupled to a first end of the first tube 902a, the first sealing feature 910a coupled to a second end of the first tube 902a, the second tube 902b coupled to the compliant structure 904 at a first end of the second tube 902b, and the second sealing feature 910b coupled to a second end of the second tube 902b.

At block 1404, the process 1400 includes sizing and/or selecting the implant device. For example, an aneurysm/aneurysmal portion of the aorta can be measured using one or more imaging techniques, such as x-rays, fluoroscopy, ultrasound, etc., to capture one or more images of the internal anatomy of a patient including the aneurysmal portion of the aorta. The one or more images can be analyzed/evaluated to determine a size/dimension of the aneurysm/aneurysmal portion, such as a length, width, diameter, or other dimension. In examples, a size/dimension of neighboring health tissue is measured/determined, such as a diameter or other dimension of the aorta within a predetermined distance to the aneurysm/aneurysmal portion.

In examples, an implant device can be cut to a length that satisfies/matches the length of the aneurysm. For instance, one or more tube portions of an implant device (which can be located on the ends of the device) can be cut to create the appropriate longitudinal length for the implant device. Additionally, or alternatively, an implant device can include multiple compliant structures coupled together via multiple tube portions (such as that shown in FIG. 6), wherein the implant device can be cut at one of the tube portions to configure the implant device with the appropriate longitudinal length.

Further, in examples, multiple implant devices are available with different sizes/dimensions so that a physician/user can select the most appropriate implant device for the patient/anatomy. To illustrate, tube portions and/or compliant structures of different implant devices can include different diameters and/or lengths such that a physician/user can select an implant device that satisfies/matches the size/dimension of the aneurysm/aneurysmal portion and/or the size/dimension of neighboring health tissue. For instance, an implant device that has tube portions with the same/smaller/similar diameter as the aorta can be selected.

At block 1406, the process 1400 includes accessing a fluid vessel that includes an enlargement. For example, a physician/user or robotics system can access an aneurysm/target site in the aorta or another fluid vessel by advancing a delivery system that includes the implant device (such as in a compressed configuration/state) to the target site. As such, the implant device can be configured to compress to a delivery/compressed configuration/state for delivery to a target site. In some cases, the delivery system is navigated/guided to the target site using one or more imaging techniques, such as using x-rays, fluoroscopy, ultrasound, etc.

In examples, the delivery system can comprise one or more catheters, sheaths, balloons, and/or other devices used to advance and/or implant the implant device, which can be disposed at least partially within the delivery system during portions of the process 1400. The implant device can be positioned within the delivery system with a first end of the implant device disposed proximally relative to the delivery system and a second end disposed distally with respect to the delivery system. In some cases, the second end that is disposed distally includes a prosthetic valve.

In examples, the delivery system comprises an outer catheter/shaft/sheath, which can be used to transport the implant device to the target implantation site. That is, the implant device can be advanced to the target implantation site at least partially within a lumen of the outer shaft, such that the implant device is held and/or secured at least partially within a distal portion of the outer shaft in a radially compressed configuration.

In examples, the delivery system comprises a tapered nosecone feature, which can facilitate advancement of the distal end of the delivery system through the tortuous anatomy of the patient and/or an outer delivery sheath or other conduit/path. The nosecone can be a separate component from the outer shaft or can be integrated with the outer shaft. In some examples, the nosecone is adjacent to and/or integrated with a distal end of the outer shaft. In some examples, the nosecone is distally tapered into a generally conical shape and can comprise and/or be formed of multiple flap-type forms that can be urged/spread apart when the implant device and/or any portions thereof, interior shafts, or devices, are advanced distally therethrough.

The delivery system can further be configured to have a guidewire disposed at least partially within the delivery system. The guidewire can provide a path through the patient from an external surface/port to the target site within the anatomy, such as from a percutaneous access point/port or natural orifice to an aneurysm/target site. In some implementations, the guidewire can pass through an interior of the implant device and/or through a lumen of a pusher device or tube of the delivery system.

At block 1408, the process 1400 includes deploying/implanting the implant device, such as from a delivery system. In examples, to deploy the implant device, an outer sheath of the delivery device is proximally pulled and/or a pusher (located proximally relative to the delivery system) is distally pushed to thereby draw the outer sheath past the distal end of the implant device, at least partially exposing/deploying the implant device. Initially the outer sheath can be withdrawn to position/attach a first sealing feature of the implant device at a first position/location within the aorta (e.g., attach the first sealing feature to a first internal portion of the aorta at one side of the aneurysm). The outer sheath can be further withdrawn to position a compliant structure of the implant device within the aneurysm. The outer sheath can be further withdrawn to position/attach a second sealing feature of the implant device at a second/proximal position/location within the aorta (e.g., attach the second sealing feature to a second internal portion of the aorta at a second side of the aneurysm). The implant device can comprise one or more radiopaque markers that can be referenced to determine/confirm the position of the implant device at various stage(s) of the process 1400 using a suitable imaging technique.

At block 1410, the process 1400 includes expanding the implant device. In some examples, one or more portions of the implant device (e.g., sealing features, a compliant structure, etc.) are self-expandable, such that implant device is expanded/fully deployed (e.g., anchored/secured to the internal tissue of the aorta) upon release from the outer sheath (as discussed above with reference to block 1408). For instance, expansion of the sealing features and/or a compliant structure can be achieved via shape memory features of the sealing features and/or a compliant structure and/or other portions of the device. To illustrate, one or more portions of the implant device (e.g., one or more frames of the device) can comprise nitinol or another shape-memory metal configured to self-expand when released from the delivery sheath/capsule.

Alternatively, or additionally, one or more portions of the implant device (e.g., the sealing features and/or a compliant structure) can be balloon/dilator expandable, such that a balloon/dilator are used to radially expand the component. The balloon/dilator can be implemented as part of or separately from the delivery system. The balloon/dilator can be inserted through the implant device to radially expand one or more portions of the implant device.

At block 1412, the process 1400 includes withdrawing the delivery system, leaving the implant device implanted. The delivery system can be withdrawn from the patient over a guidewire and/or the guidewire can be withdrawn.

As such, the process 1400 can utilize a transcatheter procedure for implantation/deployment of implant devices in accordance with aspects of the present disclosure. However, implant devices disclosed herein can be implanted using other types of minimally invasive and/or surgical procedures.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

### ADDITIONAL EXAMPLES

1. An implant device comprising: a first sealing feature configured to seal to an internal portion of a fluid vessel; a compliant structure including a frame and a covering disposed on the frame, the first sealing feature being coupled to a first end of the compliant structure, the compliant structure being configured to expand and contract radially in at least one dimension based at least in part on fluid pressure associated with the fluid vessel; and a second sealing feature coupled to a second end of the compliant structure.

2. The implant device of any example herein, in particular example 1, wherein at least one of the first sealing feature or the second sealing feature includes an expandable frame.

3. The implant device of any example herein, in particular example 2, wherein, in an expanded form, at least one of the first sealing feature or the second sealing feature includes an outer diameter that is the same as or larger than an inner diameter of a section of the fluid vessel.

4. The implant device of any example herein, in particular example 2, wherein the frame of the compliant structure and the expandable frame of at least one of the first sealing feature or the second sealing feature are integral frames.

5. The implant device of any example herein, in particular example 2, wherein the frame of the compliant structure and the expandable frame of at least one of the first sealing feature or the second sealing feature are separate frames.

6. The implant device of any example herein, in particular example 2, wherein a diameter of the compliant structure in an expanded form is the same as or larger than a diameter of the expandable frame of at least one of the first sealing feature or the second sealing feature in an expanded form.

7. The implant device of any example herein, in particular example 2, wherein the expandable frame of at least one of the first sealing feature or the second sealing feature includes a shape-memory metal.

8. The implant device of any examples herein, in particular examples 1-7, wherein a diameter of the compliant structure in an expanded form is the same as or larger than an inner diameter of a section of the fluid vessel.

9. The implant device of any examples herein, in particular examples 1-8, wherein the compliant structure is configured to be implanted within an aneurysmal section of the fluid vessel.

10. The implant device of any examples herein, in particular examples 1-9, wherein the implant device is configured to be compressed and coupled to a delivery device.

11. The implant device of any examples herein, in particular examples 1-10, further comprising: a first tube coupled to the first end of the compliant structure and including the first sealing feature; a second tube coupled to the second end of the compliant structure and including the second sealing feature; and a third tube coupled to the second end of the compliant structure and including a third sealing feature.

12. The implant device of any example herein, in particular examples 11, wherein the first tube is configured to be disposed within the fluid vessel and the second and third tubes are configured to be disposed within branches of the fluid vessel, respectively.

13. The implant device of any example herein, in particular examples 1-12, wherein the compliant structure includes: first and second arched end walls on opposite major-axis ends of the compliant structure; and first and second sidewalls on opposite minor-axis ends of the compliant structure.

14. The implant device of any example herein, in particular example 13, wherein the frame includes an oval cross-sectional shape.

15. The implant device of any example herein, in particular example 13, wherein the first and second sidewalls are deflected radially inward to form a peanut cross-sectional shape.

16. The implant device of any example herein, in particular examples 1-15, wherein at least one of the first sealing feature or the second sealing feature includes a circular cross-sectional shape and the compliant structure includes a non-circular cross-sectional shape.

17. The implant device of any example herein, in particular examples 1-16, wherein the covering is disposed on at least one of an inner or outer surface of the frame.

18. The implant device of any example herein, in particular examples 1-17, wherein the frame includes a shape-memory metal.

19. The implant device of any example herein, in particular examples 1-18, wherein the frame is a spiral frame.

20. The implant device of any example herein, in particular examples 1-19, wherein the implant device is sterilized.

21. A stent comprising: a first sealing feature configured to seal to an internal portion of a fluid vessel; a first expandable frame including a biased non-circular shape, the first sealing feature being coupled to a first end of the frame; and a second sealing feature coupled to a second end of the first expandable frame.

22. The stent of any example herein, in particular example 21, wherein the biased non-circular shape is an oval shape.

23. The stent of any example herein, in particular examples 21 or 22, wherein the biased non-circular shape is a peanut shape.

24. The stent of any example herein, in particular examples 21-23, wherein, in an expanded form, at least one of the first sealing feature or the second sealing feature includes an outer diameter that is the same as or larger than an inner diameter of a section of the fluid vessel.

25. The stent of any example herein, in particular examples 21-24, wherein at least one of the first sealing feature or the second sealing feature includes a second expandable frame.

26. The stent of any example herein, in particular example 25, wherein the first expandable frame and the second expandable frame are integral frames.

27. The stent of any example herein, in particular example 25, wherein the first expandable frame and the second expandable frame are separate frames.

28. The stent of any example herein, in particular example 25, wherein a diameter of the first expandable frame in an expanded form is the same as or larger than a diameter of the second expandable frame in an expanded form.

29. The stent of any example herein, in particular examples 21-28, wherein the first expandable frame includes a shape-memory metal.

30. The stent of any example herein, in particular examples 21-29, wherein a diameter of the first expandable frame in an expanded form is the same as or larger than an inner diameter of a healthy section of the fluid vessel.

31. The stent of any example herein, in particular examples 21-30, wherein the stent is configured to be implanted within an aneurysmal section of the fluid vessel.

32. The stent of any example herein, in particular examples 21-31, wherein the stent is configured to be compressed and coupled to a delivery system.

33. The stent of any example herein, in particular examples 21-32, further comprising: a first tube coupled to the first end of the first expandable frame and including the first sealing feature; a second tube coupled to the second end of the first expandable frame and including the second sealing feature; and a third tube coupled to the second end of the first expandable frame and including a third sealing feature.

34. The stent of any example herein, in particular example 33, wherein the first tube is configured to be disposed within the fluid vessel and the second and third tubes are configured to be disposed within branches of the fluid vessel, respectively.

35. The stent of any example herein, in particular example 21-34, wherein the first expandable frame includes: first and second arched end walls on opposite major-axis ends of the first expandable frame; and first and second sidewalls on opposite minor-axis ends of the first expandable frame.

36. The stent of any example herein, in particular example 35, wherein the first and second sidewalls are deflected radially inward to form a peanut cross-sectional shape.

37. The stent of any example herein, in particular examples 21-36, wherein at least one of the first sealing member or the second sealing member includes a circular cross-sectional shape and the first expandable frame includes a non-circular cross-sectional shape.

38. The stent of any example herein, in particular examples 21-37, wherein the covering is disposed on at least one of an inner or outer surface of the first expandable frame.

39. The stent of any example herein, in particular examples 21-38, wherein the first expandable frame includes a shape-memory metal.

40. The stent of any example herein, in particular examples 21-39, wherein the first expandable frame is a spiral frame.

41. The stent of any example herein, in particular examples 21-40, wherein the stent is sterilized.

42. A method comprising: providing an implant device that includes a first sealing feature, a compliant structure coupled to the first sealing feature, and a second sealing feature coupled to the compliant structure, the compliant structure including an expandable frame having a biased non-circular shape; accessing a fluid vessel that includes an enlargement; and implanting the implant device in the fluid vessel by: attaching the first sealing feature to a first location at a first side of the enlargement; and attaching the second sealing feature to a second location at a second side the enlargement.

43. The method of any example herein, in particular example 42, wherein accessing the fluid vessel includes advancing a delivery system to the enlargement, the delivery system including the implant device in a compressed configuration; and wherein implanting the deploying the implant device includes deploying the implant device from the delivery system.

44. The method of any example herein, in particular examples 42 or 43, further comprising: determining a size of the enlargement; determining an implant size based at least in part on the size of the enlargement; and cutting the implant device to the implant size.

45. The method of any example herein, in particular examples 42-44, wherein the expandable frame is a self-expandable frame.

46. The method of any example herein, in particular examples 42-45, wherein the enlargement is an aneurysm and the fluid vessel is a blood vessel.

47. The method of any example herein, in particular examples 42-46, wherein the fluid vessel is the aorta.

48. The method of any example herein, in particular examples 42-47, wherein the biased non-circular shape is an oval shape.

49. The method of any example herein, in particular examples 42-48, wherein the biased non-circular shape is a peanut shape.

50. The method of any example herein, in particular examples 42-49, wherein the expandable frame includes: first and second arched end walls on opposite major-axis ends of the expandable frame; and first and second sidewalls on opposite minor-axis ends of the expandable frame.

51. The method of any example herein, in particular examples 42-50, wherein at least one of the first sealing feature or the second sealing feature includes a frame.

52. The method of any example herein, in particular example 51, wherein, in an expanded form, at least one of the first sealing feature or the second sealing feature includes an outer diameter that is the same as or larger than an inner diameter of a section of the fluid vessel.

53. The method of any example herein, in particular example 51 or 52, wherein the expandable frame of the compliant structure and the frame of at least one of the first sealing feature or the second sealing feature are integral frames.

54. The method of any example herein, in particular examples 51-53, wherein the expandable frame of the compliant structure and the frame of at least one of the first sealing feature or the second sealing feature are separate frames.

55. The method of any example herein, in particular examples 51-54, wherein a diameter of the compliant structure in an expanded form is the same as or larger than a diameter of the frame of at least one of the first sealing feature or the second sealing feature in an expanded form.

56. The method of any example herein, in particular examples 51-55, wherein the frame of at least one of the first sealing feature or the second sealing feature includes a shape-memory metal.

57. The method of any example herein, in particular examples 51-56, wherein a diameter of the compliant structure in an expanded form is the same as or larger than an inner diameter of a healthy section of the fluid vessel.

### FURTHER EXAMPLES

1. A tubular graft comprising: a first tube portion configured to attach to a first portion of a fluid vessel; and a first compliant structure coupled to the first tube portion, the first compliant structure including a frame and a covering disposed on the frame, the first tube portion being coupled to a first end of the first compliant structure, the first compliant structure being configured to expand and contract radially in at least one dimension based at least in part on fluid pressure associated with the fluid vessel.

2. The tubular graft of any example herein, in particular example 1, wherein the first compliant structure includes: first and second arched end walls on opposite major-axis ends of the first compliant structure; and first and second sidewalls on opposite minor-axis ends of the first compliant structure.

3. The tubular graft of any example herein, in particular example 2, wherein the first compliant structure includes an oval cross-sectional shape.

4. The tubular graft of any example herein, in particular example 2, wherein the first and second sidewalls are deflected radially inward to form a peanut cross-sectional shape.

5. The tubular graft of any example herein, in particular examples 1-4, wherein the first tube portion is formed of a fabric.

6. The tubular graft of any example herein, in particular examples 1-5, wherein the first tube portion includes polyethylene terephthalate.

7. The tubular graft of any example herein, in particular examples 1-6, wherein the first tube portion is non-expandable.

8. The tubular graft of any example herein, in particular examples 1-7, wherein the first tube portion includes a circular cross-sectional shape and the first compliant structure includes a non-circular cross-sectional shape.

9. The tubular graft of any example herein, in particular examples 1-8, further comprising: a prosthetic valve coupled to the first tube portion.

10. The tubular graft of any example herein, in particular examples 1-9, further comprising: a second tube portion coupled to a second end of the first compliant structure and configured to attach to a second portion of the fluid vessel.

11. The tubular graft of any example herein, in particular example 10, further comprising: a second compliant structure coupled to the second tube portion and configured to expand and contract radially; and a third tube portion coupled to the second compliant structure.

12. The tubular graft of any example herein, in particular examples 1-11, further comprising: a branch tube radially-projecting from the first tube portion, the branch tube including a smaller diameter than a diameter of the first tube portion.

13. The tubular graft of any example herein, in particular examples 1-12, wherein the tubular graft is arch shaped along a length of the tubular graft.

14. The tubular graft of any example herein, in particular examples 1-13, further comprising: a second tube portion coupled to a second end of the first compliant structure; a branch tube coupled to the second tube portion; a second compliant structure coupled to the second tube portion and configured to expand and contract radially in at least one dimension; and a third tube portion coupled to the second compliant structure.

15. The tubular graft of any example herein, in particular example 14, wherein the tubular graft is arch shaped along a length of the tubular graft.

16. The tubular graft of any example herein, in particular examples 1-15, wherein a diameter of the first compliant structure in an expanded form is larger than a diameter of the first tube portion.

17. The tubular graft of any example herein, in particular examples 1-16, wherein the first compliant structure includes an oval cross-sectional shape in a contracted form and a circle cross-sectional shape in an expanded form.

18. The tubular graft of any example herein, in particular examples 1-17, wherein the covering is disposed on at least one of an inner or outer surface of the frame.

19. The tubular graft of any example herein, in particular examples 1-18, wherein the frame includes a shape-memory metal.

20. The tubular graft of any example herein, in particular examples 1-19, wherein the frame is a spiral frame.

21. The tubular graft of any example herein, in particular examples 1-20, wherein the tubular graft is sterilized.

22. A graft comprising: a first tube; a first frame including a biased non-circular shape; a covering disposed on the first frame and coupled to the first tube; and a second tube coupled to the covering.

23. The graft of any example herein, in particular example 22, wherein the biased non-circular shape is an oval shape.

24. The graft of any example herein, in particular examples 22 or 23, wherein the biased non-circular shape is a peanut shape.

25. The graft of any example herein, in particular examples 22-24, wherein at least one of the first frame or the covering includes: first and second arched end walls on opposite major-axis ends; and first and second sidewalls on opposite minor-axis ends.

26. The graft of any example herein, in particular examples 22-25, wherein at least one of the first tube or the second tube is formed of a fabric.

27. The graft of any example herein. in particular examples 22-26, wherein at least one of the first tube or the second tube is formed of polyethylene terephthalate.

28. The graft of any example herein, in particular examples 22-27, wherein at least one of the first tube or the second tube is non-expandable.

29. The graft of any example herein, in particular examples 22-28, further comprising: a prosthetic valve coupled to the first tube.

30. The graft of any example herein, in particular examples 22-29, further comprising: a second frame coupled to the second tube, the second frame including the biased non-circular shape; and a third tube coupled to the second frame.

31. The graft of any example herein, in particular examples 22-30, further comprising: a branch tube radially-projecting from the first tube, the branch tube including a smaller diameter than a diameter of the first tube.

32. The graft of any example herein, in particular examples 22-31, further comprising: a second frame including the biased non-circular shape; an additional covering disposed on the second frame; a third tube coupled to at least one of the additional covering or the second frame; and a branch tube coupled to the second tube.

33. The graft of any example herein, in particular example 32, wherein the graft is arch shaped along a length of the graft.

34. The graft of any example herein, in particular examples 22-33, wherein a diameter of the first frame in an expanded form is the same as a diameter of the first tube.

35. The graft of any example herein. in particular examples 22-34, wherein a diameter of the first frame in an expanded form is larger than a diameter of the first tube.

36. The graft of any example herein, in particular examples 22-35, wherein the covering is disposed on at least one of an inner or outer surface of the first frame.

37. The graft of any example herein, in particular examples 22-36, wherein the first frame includes a shape-memory metal.

38. The graft of any example herein, in particular examples 22-37, wherein the first frame includes a spiral frame.

39. The graft of any example herein, in particular examples 22-38, wherein the graft is sterilized.

40. A method comprising: providing a graft that includes a first tube, a first frame, a covering disposed on the first frame, and a second tube coupled to at least one of the first frame or the covering, the first frame including a biased non-circular shape; accessing a fluid vessel that includes an enlargement, a first location on one side of the enlargement, and a second location on another side of the enlargement; and implanting the graft in the fluid vessel by: attaching the first tube to the first location; and attaching the second tube to the second location.

41. The method of any example herein, in particular example 40, wherein the implanting the graft includes suturing the first tube to the first location and suturing the second tube to the second location.

42. The method of any example herein, in particular example 40 or 41, further comprising: resecting the fluid vessel at the enlargement before or after implanting the graft.

43. The method of any example herein, in particular example 42, further comprising: covering at least a portion of the graft with a portion of the fluid vessel that has been resected.

44. The method of any example herein, in particular examples 40-43, further comprising: determining a size of the enlargement; determining a graft size based at least in part on the size of the enlargement; and cutting the graft to the graft size.

45. The method of any example herein, in particular examples 40-44, wherein the enlargement is an aneurysm and the fluid vessel is a blood vessel.

46. The method of any example herein, in particular examples 40-45, wherein the fluid vessel is the aorta.

47. The method of any example herein, in particular examples 40-46, wherein the biased non-circular shape is an oval shape.

48. The method of any example herein, in particular examples 40-47, wherein the biased non-circular shape is a peanut shape.

49. The method of any example herein, in particular examples 40-48, wherein the first frame includes: first and second arched end walls on opposite major-axis ends of the first frame; and first and second sidewalls on opposite minor-axis ends of the first frame.

50. The method of any example herein, in particular examples 40-49, wherein at least one of the first tube or the second tube is formed of a fabric.

51. The method of any example herein, in particular examples 40-50, wherein at least one of the first tube or the second tube is formed of polyethylene terephthalate.

52. The method of any example herein, in particular examples 40-51, wherein at least one of the first tube or the second tube is non-expandable.

53. The method of any example herein, in particular examples 40-52, wherein the graft includes a prosthetic valve coupled to the second tube.

54. The method of any example herein, in particular example 53, wherein implanting the graft includes implanting the prosthetic valve at a native valve associated with the fluid vessel.

55. The method of any example herein, in particular examples 40-54, wherein the graft further includes a second frame adjacent to the second tube, a third tube adjacent to the second frame, a branch tube coupled to the second tube, the second frame including the biased non-circular shape.

56. The method of any example herein, in particular examples 40-55, wherein the graft is arch shaped along a length of the graft.

Depending on the example, certain acts, events, or functions of any of the processes or algorithms described herein can be performed in a different sequence, can be added, merged, and/or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that some examples include, while other examples do not include, certain features, elements, and/or steps. Thus, such conditional language is not generally intended to imply that features, elements, and/or steps are in any way required for one or more examples or that one or more examples necessarily include logic for deciding, with or without author input or prompting, whether these features, elements, and/or steps are included or are to be performed in any particular example. The terms “comprising,” “including,” “having,” and the like are generally synonymous, used in their ordinary sense, and used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is understood with the context as used in general to convey that an item, term, element, etc. can be either X, Y, or Z. Thus, such conjunctive language is not generally intended to imply that certain examples require at least one of X, at least one of Y, and at least one of Z to each be present.

In examples, various features are sometimes grouped together in a single example, figure, or description thereof for the purpose of streamlining the disclosure and aiding in the understanding of one or more of the various aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the subject matter herein disclosed and claimed below should not be limited by the particular examples described herein.

Certain ordinal terms (e.g., “first” or “second”) may be provided for ease of reference and do not necessarily imply physical characteristics or ordering. Therefore, as used herein, an ordinal term (e.g., “first,” “second,” “third,” etc) used to modify an element, such as a structure, a component, an operation, etc., does not necessarily indicate priority or order of the element with respect to any other element, but rather can generally distinguish the element from another element having a similar or identical name (but for use of the ordinal term). In addition, as used herein, indefinite articles (“a” and “an”) can indicate “one or more” rather than “one.” Further, an operation performed “based on” a condition or event can also be performed based on one or more other conditions or events not explicitly recited.

Unless otherwise defined, terms (including technical and/or scientific terms) used herein can have the same meaning as commonly understood by one of ordinary skill in the art to which examples belong. Terms, such as those defined in commonly used dictionaries, can be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, can be used herein for ease of description to describe the relations between one element or component and another element or component as illustrated in the drawings. Spatially relative terms can encompass different orientations of the device in use or operation, in addition to the orientation depicted in the drawings. For example, in the case where a device shown in the drawing is turned over, the device positioned “below” or “beneath” another device can be placed “above” another device. Accordingly, the illustrative term “below” can include both the lower and upper positions. The device can also be oriented in the other direction, and thus the spatially relative terms can be interpreted differently depending on the orientations.

Unless otherwise expressly stated, comparative and/or quantitative terms, such as “less,” “more.” “greater,” and the like, can encompass the concepts of equality. For example, “less” can mean not only “less” in the strictest mathematical sense, but also “less than or equal to.”

What is claimed is:

1. A tubular graft for replacing a segment of a fluid vessel, the tubular graft comprising:
a first tube portion adapted to attach to a first resected portion of the fluid vessel with a first attachment element;
a second tube portion adapted to attach to a second resected portion of the fluid vessel with a second attachment element; and
a first compliant structure coupled between the first tube portion and the second tube portion, the first compliant structure including a frame and a covering disposed on the frame, the first compliant structure being adapted to expand and contract radially in at least one dimension based at least in part on fluid pressure associated with the fluid vessel, the first compliant structure including first and second arched end walls on opposite major-axis ends of the first compliant structure and first and second sidewalls on opposite minor-axis ends of the first compliant structure.

2. The tubular graft of claim 1, wherein the first compliant structure includes an oval cross-sectional shape.

3. The tubular graft of claim 1, wherein the first and second sidewalls are deflected radially inward to form a peanut cross-sectional shape.

4. The tubular graft of claim 1, wherein at least one of the first tube portion or the second tube portion is formed of a fabric. 5 The tubular graft of claim 1, wherein at least one of the first tube portion or the second tube portion includes polyethylene terephthalate.

6. The tubular graft of claim 1, wherein at least one of the first tube portion or the second tube portion is non-expandable.

7. A tubular graft comprising:
a first tube portion adapted to attach to a first portion of a fluid vessel, the first tube portion including a circular cross-sectional shape; and
a first compliant structure coupled to the first tube portion, the first compliant structure including a frame and a covering disposed on the frame, the first tube portion being coupled to a first end of the first compliant structure, the first compliant structure being adapted to expand and contract radially in at least one dimension based at least in part on fluid pressure associated with the fluid vessel, the first compliant structure adapted to bias towards a non-circular cross-sectional shape.

8. The tubular graft of claim 7, further comprising:
a prosthetic valve coupled to the first tube portion.

9. The tubular graft of claim 7, further comprising:
a second tube portion coupled to a second end of the first compliant structure and adapted to attach to a second portion of the fluid vessel.

10. The tubular graft of claim 9, further comprising:
a second compliant structure coupled to the second tube portion and adapted to expand and contract radially; and
a third tube portion coupled to the second compliant structure.

11. The tubular graft of claim 7, further comprising:
a branch tube radially projecting from the first tube portion, the branch tube including a smaller diameter than a diameter of the first tube portion.

12. The tubular graft of claim 7, wherein the tubular graft is arch shaped along a length of the tubular graft.

13. The tubular graft of claim 7, further comprising:
a second tube portion coupled to a second end of the first compliant structure;
a branch tube coupled to the second tube portion;
a second compliant structure coupled to the second tube portion and adapted to expand and contract radially in at least one dimension; and
a third tube portion coupled to the second compliant structure.

14. The tubular graft of claim 7, wherein the tubular graft is arch shaped along a length of the tubular graft.

15. A tubular graft comprising:
a tube portion adapted to attach to a first resected portion of a fluid vessel; and
a compliant structure adapted to replace and provide compliance to a section of the fluid vessel, the compliant structure including a frame and a covering disposed on the frame, the compliant structure being adapted to expand radially in a first dimension while contracting radially in a second dimension based at least in part on fluid pressure associated with the fluid vessel.

16. The tubular graft of claim 15, wherein a diameter of the compliant structure in an expanded form is larger than a diameter of the tube portion.

17. The tubular graft of claim 15, wherein the covering is disposed on at least one of an inner or outer surface of the frame.

18. The tubular graft of claim 15, wherein the frame includes a shape-memory metal.

19. The tubular graft of claim 15, wherein the frame is a spiral frame.

20. The tubular graft of claim 15, wherein the compliant structure includes an oval cross-sectional shape in a contracted form and a circle cross-sectional shape in an expanded form.
